(19) United States
(12) Patent Application Publication
(10) Pub. No.: US 2025/0041272 A1
(43) Pub. Date: Feb. 6, 2025

(54) COMPOSITIONS AND METHODS FOR TREATING CANCER VIA PTP1B INHIBITION

(71) Applicant: The University of North Carolina at Chapel Hill, Chapel Hill, NC (US)

(72) Inventor: Eric Ubil

(21) Appl. No.: 18/706,869

(22) PCT Filed: Nov. 3, 2022
(86) PCT No.: PCT/US2022/048796

§ 371 Date: May 2, 2024

Related U.S. Application Data
(60) Provisional application No. 63/275,216, filed on Nov. 3, 2021.

(51) Int. Cl. A61K 31/404; A61K 33/243; A61K 45/06; A61P 35/00

(52) U.S. Cl. CPC A61K 31/404; A61K 33/243; A61K 45/06; A61P 35/00

(57) ABSTRACT

Provided are pharmaceutical compositions for treating cancer patients. The pharmaceutical compositions include a combination of compounds inhibiting PTP1 b activity and chemotherapeutic agents. The PTP 1 b inhibitors in such pharmaceutical compositions can include a small molecule such as an anti-sense inhibitor, an allosteric/reversible inhibitor, and/or a permanent/chemical modification inhibitor. Methods of treating subjects are also provided. Such treatment methods include administering to a subject in need of treatment a pharmaceutical composition including one or more compounds inhibiting PTP1b activity and one or more chemotherapeutic agents. Also provided herein are methods of restoring an anti-tumor immune response in a subject receiving chemotherapy, including administering to the subject a PTP 1 b inhibitor prior to, during and/or after receiving a chemotherapeutic agent, whereby an anti-tumor immune response in the subject is restored and/or enhanced.

### CROSS-REFERENCE TO RELATED APPLICATIONS

This application claims benefit of and priority to U.S. Provisional Patent Application Ser. No. 63/275,216, filed Nov. 3, 2021, herein incorporated by reference in its entirety.

### GRANT STATEMENT

This invention was made with government support under Grant No. CA205398 awarded by the National Institutes of Health. The government has certain rights in the invention.

### TECHNICAL FIELD

The presently disclosed subject matter is directed to methods, treatments and compositions for cancer therapy and related conditions. More particularly, the presently disclosed subject matter is directed to restoration of the anti-tumor immune response during chemotherapy by PTP1 b inhibition via therapeutic compositions.

### BACKGROUND

By suppressing the anti-tumor immune response, cancer cells foster a more permissive milieu for their continued growth and for disease progression. Some clinically relevant immune suppressive mechanisms utilized by tumors are adaptive immune checkpoints, like PD-1/PD-L1 (1-3) and CTLA4 (4, 5), which dampen the ability of T cells to eliminate tumors. Adaptive immune checkpoint blockade is a widely used therapeutic modality, with 30% of all cancer patients receiving some form of adaptive immunotherapy (6). While a boon to many patients, most that receive T-cell directed immunotherapy will either not respond or will later relapse. In the case of melanoma, which is generally more responsive to checkpoint blockade than other cancers due to its high mutational burden, patients receiving combination adaptive immunotherapy (Nivolumab plus Ipilimumab) have an average 5-year survival of 52% and a complete response rate of 22% (7). Unfortunately, patients with other forms of cancer, including prostate, breast, or colon, are almost entirely unresponsive (8).

For many patients, the next best option is chemotherapy. In addition to killing cancerous cells directly, chemotherapy induces immunogenic tumor cell death by causing the uncontrolled release of tumor neo-antigens and Damage Associated Molecular Patterns (DAMPs). While the mutational burden of tumors dictates the quantity of neo-antigens available for T cell recognition, DAMPs are a near-universal aspect of dead or dying cells and directly stimulate an innate pro-inflammatory response. Known DAMPs include transcription factors (e.g., HMGB1, HMGN1 (9-12)), stress-related proteins (e.g., Hsp60/70/90 (13-15)), nucleic acids(16-18), and other factors (19, 20) that bind and activate pro-inflammatory signaling complexes such as the Toll-like Receptor (TLR) family (21-24).

Tumor associated macrophages, which are often one of the most populous intra-tumoral immune subsets (25, 26), can adopt a spectrum of activation states ranging from pro-inflammatory (M1) to pro-wound healing (M2). These innate immune cells are of clinical importance because intra-tumoral infiltration of M2 macrophages has been associated with worse cancer patient outcomes (27-29). Current macrophage targeted cancer treatments involve either reducing the number of macrophages within the tumor, thereby reducing M2 contributions to tumor progression, or shifting polarization state away from the M2 phenotype (reviewed in (30)).

Much like T cell inactivating mechanisms, tumors utilize innate immune checkpoints to prevent the macrophage pro-inflammatory anti-tumor response. Thus, there remains a need for therapeutic compositions and approaches to combat cancer immune suppressive mechanisms to thereby enhance the efficacy of existing cancer therapies.

As disclosed herein, it was discovered that Mer activation by tumor cells dramatically reduces the macrophage response during chemotherapy. It was found that tumor secretions activate Mer, leading to reduced macrophage expression of the key TLR adapter protein MyD88, which is essential for macrophages to respond to chemotherapy-released DAMPs. Further, it is shown herein that this process is a central mechanism utilized by several tumor types (including melanoma, lung, breast, and pancreatic cancers) to abrogate the macrophage response to most TLR agonists and effectively mitigate the innate immune response. As disclosed herein, PTP1b inhibition surprisingly prevents Mer-mediated suppression and co-treatment of tumor-bearing mice with a PTP1 b inhibitor and chemotherapy dramatically improves survival in multiple chemotherapy resistant preclinical models.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

Provided in some embodiments are pharmaceutical compositions comprising one or more compounds inhibiting PTP1 b activity, and one or more chemotherapeutic agents. In some embodiments, the PTP1 b inhibitor comprises a small molecule, optionally wherein the PTP1b inhibitor comprises an anti-sense inhibitor, an allosteric/reversible inhibitor, and/or a permanent/chemical modification inhibitor. In some embodiments, the PTP1b inhibitor is selected from the group consisting of BVT948, PTP1b inhibitor III, NSC87877, PTP1b inhibitor, PTP Inhibitor IV, MSI-1436 and ISIS-PTP1B_{RX}. In some embodiments, the PTP1b inhibitor is BVT948.

In some embodiments, PTP1b inhibition by one or more compounds inhibiting PTP1b activity substantially prevents Pros1:Mer:Stat1-mediated suppression of MyD88 expression, optionally wherein the Pros1:Mer:Stat1-mediated suppression of MyD88 expression is prevented at least about 40% to at least about 99%. In some embodiments, the composition rescues the macrophage M1 response to tumor DAMPs due to PTP1 b inhibition.

In some embodiments, the one or more chemotherapeutic agents comprise an alkylating agent, a nitrosourea, an anti-metabolite, a plant alkaloid/natural product, an anti-tumor antibiotic and/or combination thereof, optionally wherein the chemotherapeutic agent is selected from the group consisting of daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxu-ridine (5-FUdR), methotrexate (MTX), colchicine, Vincristine, vinblastine, etoposide, teniposide, cisplatin and diethylstilbestrol (DES).

In some embodiments, the composition improves a patient's responsiveness to chemotherapy upon administration to a patient. In some embodiments, the composition is configured to provide a co-treatment of tumors and cancers with chemotherapy and PTP1 b inhibition to restore the macrophage DAMP pro-inflammatory response. In some embodiments, the compositions further comprise a pharmaceutically acceptable carrier, diluent, enhancer, excipient, and/or combinations thereof. In some embodiments, the compositions further comprise a checkpoint blockade component, optionally wherein the checkpoint blockade component comprises an anti-PD-1, anti-PD-L1, or anti-CTLA4 component, or combination thereof.

Also provided herein in some embodiments are methods of treating a subject, the methods comprising administering to a subject in need of treatment a pharmaceutical composition comprising one or more compounds inhibiting PTP1b activity, and one or more chemotherapeutic agents. In some embodiments, the subject is a subject suffering from a cancer or tumor, optionally where the subject is suffering from a cancer or tumor type selected from the group consisting of melanoma, lung cancer, pancreatic cancer and/or breast cancer. In some embodiments, the subject is a human subject, optionally wherein the subject is a human subject suffering from a chemotherapy resistant tumor or cancer.

In some embodiments, the PTP1b inhibitor comprises a small molecule, optionally wherein the PTP1 b inhibitor comprises an anti-sense inhibitor, an allosteric/reversible inhibitor, and/or a permanent/chemical modification inhibitor. In some embodiments, the PTP1b inhibitor is selected from the group consisting of BVT948, PTP1b inhibitor III, NSC87877, PTP1b inhibitor, PTP Inhibitor IV, MSI-1436 and ISIS-PTP1B_{RX}. In some embodiments, the PTP1 b inhibitor is BVT948.

In some embodiments, PTP1b inhibition by one or more compounds inhibiting PTP1b activity substantially prevents Pros1:Mer:Stat1-mediated suppression of MyD88 expression, optionally wherein the Pros1:Mer:Stat1-mediated suppression of MyD88 expression is prevented at least about 40% to at least about 99%. In some embodiments, the composition rescues the macrophage M1 response to tumor DAMPs due to PTP1b inhibition. In some embodiments, the one or more chemotherapeutic agents comprise an alkylating agent, a nitrosourea, an anti-metabolite, a plant alkaloid/natural product, an anti-tumor antibiotic and/or combination thereof, optionally wherein the chemotherapeutic agent is selected from the group consisting of daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxu-ridine (5-FUdR), methotrexate (MTX), colchicine, Vincristine, vinblastine, etoposide, teniposide, cisplatin and diethylstilbestrol (DES). In some embodiments, the method substantially prevents in the subject a suppression of an immune response by PTP1b inhibition, and wherein macrophage responsiveness to tumors DAMPs is restored, optionally wherein the prevention in the suppression of an immune response by PTP1 b is about 40% to about 100%.

In some embodiments, the PTP1b inhibitor is co-administered with the chemotherapeutic agent. In some embodiments, the co-administration of the PTP1 b inhibitor with the chemotherapeutic agent has a synergistic effect causing an improved outcome as compared to the administration of the PTP1 b inhibitor or the chemotherapeutic agent alone, optionally wherein the improved outcome comprises an about 25% to about 100% reduction in tumor growth relative to the administration of the PTP1b inhibitor or the chemotherapeutic agent alone. In some embodiments, administration of the PTP1 b inhibitor improves the patient's responsiveness to chemotherapy, optionally an improvement of at least 50% as compared to chemotherapy without PTP1b inhibition. In some embodiments, administration of the PTP1 b inhibitor improves the patient's responsiveness to chemotherapy, wherein the patient is suffering from a chemotherapy resistant tumor or cancer.

Provided in some embodiments are methods of restoring an anti-tumor immune response in a subject receiving chemotherapy, the methods comprising administering to the subject a PTP1b inhibitor prior to, during and/or after receiving a chemotherapeutic agent, wherein an anti-tumor immune response in the subject is restored and/or enhanced. In some embodiments, the subject is a subject suffering from a cancer or tumor, optionally a subject suffering from a tumor or cancer type selected from the group consisting of melanoma, lung cancer, pancreatic cancer and/or breast cancer. In some embodiments, the chemotherapeutic agent comprises an alkylating agent, a nitrosourea, an anti-metabolite, a plant alkaloid/natural product, an anti-tumor antibiotic and/or combination thereof, optionally wherein the chemotherapeutic agent is selected from the group consisting of daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxu-ridine (5-FUdR), methotrexate (MTX), colchicine, Vincristine, vinblastine, etoposide, teniposide, cisplatin and diethylstilbestrol (DES). In some embodiments, administration of the PTP1 b inhibitor improves the subject's responsiveness to the chemotherapy, optionally an improvement of at least about 50% as compared to chemotherapy without PTP1 b inhibition. In some embodiments, such methods further comprise administering to the subject a checkpoint blockade component, optionally wherein the checkpoint blockade component comprises an anti-PD-1, anti-PD-L1, or anti-CTLA4 component, or combination thereof.

These and other objects are achieved in whole or in part by the presently disclosed subject matter. Other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1. Tumor secretions suppress macrophage expression of pro-inflammatory pathway components including the TLR adapter MyD88. (A) Heat map analysis of RNAseq data comparing gene expression of untreated or IFNγ and LPS stimulated SC macrophages cultured in the presence or absence of MB-MDA-231 breast cancer cells for 24 hours (n=3). (B) Reactome analysis of M1 associated pathways downregulated by MB-MDA-231 cells (n=3 as described in A). *P<0.05 means significant Reactome enrichment. Transwell co-culture of melanoma, lung, pancreatic or breast cancer lines suppress IFNγ and LPS induced MyD88 (C) mRNA (as measured by qRT-PCR, n=5) and (D) protein expression (measured by Western blot, n=4) in C57BL/6J macrophages. (E) Immunofluorescent staining and colocalization analysis of changes of MyD88 expression in Lyz2-Cre:tdTomato labeled M1 induced macrophages cultured with B16F10 conditioned medium for 24 hours (n=3, scale bar=50 μm). (F) Higher MyD88 expression is associated with improved survival in TCGA data for melanoma patients (n=115 for both high and low). Data are mean+/−SEM; *P<0.05 relative to untreated, †P<0.05 relative to M1 induced. P values calculated by 2-tailed Student's t test.

FIG. 2. Tumor suppression of macrophage MyD88 is associated with decreased pro-inflammatory signaling during TLR agonism. (A) TLR expression of naïve macrophages as measured by qRT-PCR (n=5). (B) After 24 hours of the described treatment, TLR4 expression is the only TLR suppressed by tumor secretions based on qRT-PCR analysis (n=5). (C) Tumor mediated suppression of TLR agonist induced macrophage MyD88 is associated with reduced pro-inflammatory gene expression measured by qRT-PCR (n=5). Data are mean+/−SEM; *P<0.05 relative to untreated, †P<0.05 relative to M1 induced. P values calculated by 2-tailed Student's t test.

FIG. 3. Diverse tumors suppress macrophage Stat1 activation which leads to reduced MyD88 expression. (A) Transwell co-culture of cancer cell lines with M1 induced macrophages leads to reduced phospho-and total-Stat1 levels as determined by Western blot analysis (n=3). (B) Immunofluorescent staining shows reduced total and nuclear of Stat1 when Lyz2-Cre:tdTomato IFNγ and LPS treated macrophages are cultured in presence of B16F10 conditioned medium (n=4, scale bar=50 μm). (C) ChIP-qPCR measurement of Stat1 binding to the proximal MyD88 promoter in M1 macrophages is reduced by transwell co-culture with B16F10 cells (n=2). (D) qRT-PCR comparison of MyD88 expression levels of IFNγ and LPS treated macrophages either treated with Stat1 inhibitor (Fludarabine) or transwell co-cultured with B16F10 cells (n=6). (E) Plot comparing survival of melanoma patients with high or low Stat1 expression (n=229 for both high and low). (F) Co-expression analysis of TCGA data describing correlation between Stat1 and MyD88 in melanoma patient tumors (n=229). Data are mean+/−SEM; *P<0.05 relative to untreated, †P<0.05 relative to M1 induced. P values calculated by 2-tailed Student's t test.

FIG. 4. PTP1b inhibition prevents Pros1:Mer:Stat1-mediated suppression of MyD88 expression. (A) Tumor reduced pro-inflammatory macrophage Stat1 activation and MyD88 expression are restored via PTP1b inhibition at 24 hours as determined by Western blot analysis (n=3). (B) PTP1 b inhibition rescues MyD88 expression of pro-inflammatory macrophages co-cultured with B16F10 melanoma cells for 24 hours (n=5). (C) Immunofluorescent staining shows changes in MyD88 protein levels in naïve and pro-inflammatory macrophages either suppressed by B16F10 conditioned medium or rescued with PTP1 b inhibition (BVT948) after 24 hours (n=4, white arrowheads indicate areas of MyD88 expression, scale bar=50 μm). (D) Ternary complex formation of PTP1 b with Mer and Stat1 as measured by immunoprecipitation during co-culture of IFNγ and LPS stimulated macrophages with B16F10 tumor cells in the presence or absence of PTP1b inhibition (BVT948). (E) Co-immunoprecipitation of ternary complex members associated with Stat1 during tumor suppression and PTP1b inhibition rescue after 24 hours (n=3). (F) Tumor mediated suppression of MyD88 in Axl- but not Mer- or Tyro3-KO macrophages after 24 hours co-culture as determined by qRT-PCR (n=5). (G) Relative TAM ligand expression of B16F10 tumor cells by qRT-PCR (n=5). (H) Exogenous addition of 1.5 ug/ml Recombinant murine Pros1 suppresses macrophage MyD88 expression similarly to transwell co-culture with B16F10 cells (n=4). (I) siRNA knock-down of Pros1, but not Gas6, in B16F10 tumor cells restores MyD88 expression in transwell co-cultured pro-inflammatory macrophages (n=4). Data are mean+/−SEM; *P<0.05 relative to untreated, †P<0.05 relative to M1 induced. P values calculated by 2-tailed Student's t test.

FIG. 5. PTP1b inhibition rescues the macrophage M1 response to tumor DAMPs. (A) B16F10 tumor cells were serum and amino acid starved for 3 days, conditioned medium 0.2 μm filter sterilized and applied to macrophages with or without BVT948 treatment and pro-inflammatory gene expression measured by qRT-PCR after 24 hours (n=5). (B) Filtered starved GEMM6 tumor cell conditioned medium treated with either Proteinase K or Benzonase for 4 hours was applied to macrophages either treated with BVT or without for 24 hours and gene expression measured by qRT-PCR (n=5). Mice bearing subcutaneous GEMM6 tumors received either vehicle or PTP1 b inhibitor (BVT948) monotherapy when tumors became palpable (50 mm³) and tumor volume measured (n=8 mice per treatment arm). For (A) *P<0.05 relative to untreated, †P<0.05 relative to B16F10 CM serum starvation; for (B) *P<0.05 relative to B16F10 CM serum starvation, †P<0.05 relative to B16F10 CM serum starvation+BVT948; P values calculated by 2-tailed Student's t test.

FIG. 6. Co-treatment of tumors with chemotherapy and PTP1 b inhibition restores the macrophage DAMP pro-inflammatory response and leads to improve outcomes in preclinical models. (A) qRT-PCR measurement of pro-inflammatory gene expression in Cisplatin treated transwell co-cultured macrophages and B16F10 cells in the presence or absence of PTP1b inhibition (BVT948) (n=8). (B) M1 associated gene expression cannot be restored by PTP1b inhibition in MyD88 knockout macrophages (n=8). (C) Mer deficient macrophages do not require PTP1b inhibition to become activated during Cisplatin treatment (n=5). Tumor growth of subcutaneously implanted (D) B16F10, (E) GEMM6 or (F) LLC when treated with vehicle, chemotherapy indicated, or combination therapy (n=8 mice per study arm). Data are mean+/−SEM; For (A,B,C) *P<0.05 relative to untreated, †P<0.05 relative to M1 induced. P values calculated by 2-tailed Student's t test; For (D,E,F) *P<0.05 relative to vehicle treated. P values calculated by two-way ANOVA using Tukey as post-hoc test.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### 1. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments 1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

As used herein, the term “adjuvant” refers to a substance that elicits an enhanced immune response when used in combination with a specific antigen.

As use herein, the terms “administration of” and/or “administering” a compound, therapeutic composition or pharmaceutical composition should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “aqueous solution” as used herein can include other ingredients commonly used, such as sodium bicarbonate described herein, and further includes any acid or base solution used to adjust the pH of the aqueous solution while solubilizing a peptide.

The term “biological sample”, as used herein, refers to samples obtained from a subject, including but not limited to skin, hair, tissue, blood, plasma, cells, sweat, and urine.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The terms “treatment” and “treating” as used herein refer to both therapeutic treatment and prophylactic or preventative measures, wherein the object is to prevent or slow down (lessen) the targeted pathologic condition (e.g. cancer), prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fv, single chain Fv, complementarity determining regions (CDRs), VL (light chain variable region), VH (heavy chain variable region), Fab, F(ab′)₂ and any combination of those or any other functional portion of an immunoglobulin peptide capable of binding to target antigen.

### II. Subjects

The subject to be treated is desirably a human subject, although it is to be understood that the principles of the disclosed subject matter indicate that the compositions and methods are effective with respect to invertebrate and to all vertebrate species, including mammals, which are intended to be included in the term “subject”. Moreover, a mammal is understood to include any mammalian species in which screening is desirable, particularly agricultural and domestic mammalian species.

The disclosed methods are particularly useful in the treatment of warm-blooded vertebrates. Thus, the presently disclosed subject matter concerns mammals and birds.

More particularly, provided herein is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, provided herein is the treatment of livestock, including, but not limited to, domesticated swine (pigs and hogs), ruminants, horses, poultry, and the like.

In some embodiments, the subject to be used in accordance with the presently disclosed subject matter is a subject in need of treatment. In some embodiments, a subject can have or be believed to have a cancer, tumor or related condition.

### III. Pharmaceutical Compositions and Methods of Treatment

The ability to suppress the immune response is characteristic of many cancers. While adaptive immune checkpoint directed therapies have improved outcomes for many patients, it is clear that only a subset will respond, necessitating continued identification and targeting of other tumor-suppressed immune mechanisms. Here we describe an innate immune checkpoint utilized by diverse tumor types (melanoma, lung, pancreatic, breast) to suppress the immune response during chemotherapy. Tumor secreted Pros1 binds to the macrophage Mer receptor, initiating ternary complex formation between the phosphatase PTP1b and Stat1, leading to reduced Stat1 activation. By suppressing Stat1 activity, tumors cause reduced macrophage expression of the key Toll-Like Receptor signaling transduction protein, MyD88. When Damage Associated Molecular Patterns activate Toll-Like Receptors, MyD88 is essential for downstream pro-inflammatory activation. By effectively downregulating MyD88, tumors prevent macrophage activation during chemotherapy, thereby fostering a more tumor-permissive environment. We found that by combining PTP1b inhibition with chemotherapy, we can rescue the macrophage pro-inflammatory response. Further, in multiple preclinical models, combination therapy led to an approximately 50% reduction in tumor growth. Since chemotherapy is a widely used treatment modality, our discovery has tremendous potential to improve patient outcomes.

Generally, the present disclosure relates to compounds, compositions and methods for modulating the activity and/or biological activity of PTP1b and/or intermediates in the same biological pathway. Such modulation can be used in some aspects for the treatment of disease, e.g. cancer.

Provided in some embodiments are pharmaceutical compositions comprising one or more compounds inhibiting PTP1 b activity, and one or more other chemotherapeutic agents. In some embodiments, the PTP1b inhibitor comprises, but is not limited to, a small molecule, optionally an anti-sense molecule or inhibitor (e.g. ISIS version), an allosteric/reversible inhibitor (most common examples), and/or a permanent/chemical modification inhibitor (e.g. BVT948). More particularly, in some embodiments, and by way of example only and not limitation, the PTP1b inhibitor includes BVT948, PTP1b inhibitor III, NSC87877, PTP1b inhibitor, PTP Inhibitor IV, MSI-1436 (Trodusquemine), and/or ISIS-PTP1B_{RX}, or any other suitable inhibitor. In some preferred embodiments the PTP1b inhibitor can be BVT948, or similar inhibitor. In some embodiments, the PTP1b inhibitors can be further defined as follows: ISIS-PTP1B_{RX} (anti-sense inhibitor); PTP inhibitor, PTP inhibitor III, PTP inhibitor IV (allosteric/reversible, cell-permeable protein tyrosine phosphatase inhibitor); MSI-1436 (selective, allosteric, non-competitive inhibitor of PTP1b); and BVT948 (irreversible, noncompetitive, cell-permeable protein tyrosine phosphatase inhibitor). In some aspects, the PTP1b inhibition the by one or more compounds inhibiting PTP1b activity prevents Pros1:Mer:Stat1-mediated suppression of MyD88 expression. To elaborate, in some aspects, the Pros1:Mer:Stat1-mediated suppression of MyD88 expression is prevented by at least about 40% to at least about 99%, or more, optionally at least about 50% to at least about 99%, optionally at least about 60% to at least about 99%, optionally at least about 70% to at least about 99%, optionally at least about 80% to at least about 99%. In doing so, the composition effectively rescues the macrophage M1 response to tumor DAMPs due to PTP1b inhibition.

In some embodiments, the disclosed pharmaceutical composition can include one or more chemotherapeutic agents in addition to the PTP1b inhibitor, where the chemotherapeutic agent can be, for example, an alkylating agent (e.g., Cisplatin, Carboplatin, Cyclophosphamide), a nitrosourea (e.g., Carmustine, Lomustine), an anti-metabolite (e.g., 5-Fluorouracil, Methotrexate, Fludarabine), a plant alkaloids/natural product (e.g., Paclitaxel), an anti-tumor antibiotic (e.g., Doxorubicin), and/or combinations thereof. More particularly, the chemotherapeutic agent can be, by way of example and not limitation, daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxu-ridine (5-FUdR), methotrexate (MTX), colchicine, Vincristine, vinblastine, etoposide, teniposide, cisplatin and diethylstilbestrol (DES). See, generally, The Merck Manual of Diagnosis and Therapy, 15th Ed., Berkow et al., eds., 1987, Rahway, N.J., pages 1206-1228).

The disclosed pharmaceutical compositions can in some embodiments be configured to improve a patient's responsiveness to chemotherapy. For example, the composition can be configured to provide a co-treatment of tumors and cancers with chemotherapy and PTP1 b inhibition to restore the macrophage DAMP pro-inflammatory response. In some aspects, the pharmaceutical compositions can comprise a pharmaceutically acceptable carrier, diluent, enhancer, excipient, and/or combinations thereof.

In some embodiments, the disclosed pharmaceutical compositions can further comprise a checkpoint blockade component, optionally wherein the checkpoint blockade component comprises an anti-PD-1, anti-PD-L1, or anti-CTLA4 component, or combination thereof. More particularly, in some embodiments, the checkpoint blockade is achieved using antibodies. By way of example and not limitation, such checkpoint blockades, or antibodies, include anti-PD-1 antibodies: Pembrolizumab, Nivolumab, Cemiplimab; anti-PD-L1 antibodies: Atezolizumab, Durvalumab, Avelumab; and anti-CTLA4 antibodies: Ipilimumab.

The disclosed pharmaceutical compositions can be in a single formulation, or optionally in separate formulations to be administered simultaneously or in any sequence. That is, the PTP1b inhibitor can be administered before, during or after administration of the chemotherapeutic agent, or any combination thereof.

Also provided herein are methods of treating a subject. Such methods can in some embodiments comprise administering to a subject in need of treatment any of the pharmaceutical compositions disclosed herein. The subjects treated can be suffering from a cancer or tumor, optionally a subject suffering from a tumor or cancer type selected from melanoma, lung, pancreatic and/or breast. In some embodiments, the subject can be a human subject, optionally wherein the subject is a human subject suffering from a chemotherapy resistant tumor or cancer. The treatment methods prevent in the subject a suppression of an immune response by PTP1 b inhibition, and wherein macrophage responsiveness to tumors DAMPs is restored. More particularly, in some aspects macrophage responsiveness to tumors DAMPs is restored such that the prevention in the suppression of an immune response by PTP1b is about 40% to about 100%, or about 50% to about 90%, or about 60% to about 80%.

The methods comprise in some embodiments the co-treatment of a tumor or cancer with chemotherapy and PTP1 b inhibition. The co-administration of the PTP1 b inhibitor with the chemotherapeutic agent has a synergistic effect causing an improved outcome as compared to the administration of the PTP1b inhibitor or the chemotherapeutic agent alone. The improved outcome can in some aspect comprise an about 25% to about 100% reduction in tumor growth relative to the administration of the PTP1b inhibitor or the chemotherapeutic agent alone. That is, the co-treatment can cause a synergistic improvement of about 25% to about 100%, about 40% to about 100%, about 60% to about 100%, or about 80% to about 100% reduction in tumor growth relative to monotherapy alone.

In some embodiments, such methods can further comprise administering to the subject a checkpoint blockade component, optionally wherein the checkpoint blockade component comprises an anti-PD-1, anti-PD-L1, or anti-CTLA4 component, or combination thereof. Such checkpoint blockade components can comprise any suitable antibody or immunogenic agent having anti-PD-1, anti-PD-L1, or anti-CTLA4 activity or binding affinity.

In some embodiments, and as the data herein show, the administration of the PTP1 b inhibitor can improve the patient's responsiveness to chemotherapy, in some aspects an improvement of at least about 10%, about 20%, about 30%, about 40%, about 50%, or more, as compared to chemotherapy without PTP1 b inhibition. In some aspects, the administration of the PTP1b inhibitor improves the patient's responsiveness to chemotherapy, wherein the patient is suffering from a chemotherapy resistant tumor or cancer. In some embodiments, chemotherapy resistance involves innate or acquired resistance of tumors/tumor cells to chemotherapy. Clinically, this would be manifested as a lack of response or a diminished responsiveness of the patient to chemotherapy treatment, e.g. at least an about 10%, 20%, 30%, 50%, 75% or more reduction in responsiveness.

The compounds disclosed herein can be formulated in accordance with the routine procedures adapted for a desired administration route. Accordingly, in some embodiments, the presently disclosed subject matter provides a pharmaceutical composition comprising a therapeutically effective amount of a compound as disclosed hereinabove (e.g., a PTP1b inhibitor, alone or combined with a chemotherapeutic agent), or a pharmaceutically acceptable salt or solvate thereof, and a pharmaceutically acceptable carrier. The therapeutically effective amount can be determined by testing the compounds in an in vitro or in vivo model and then extrapolating therefrom for dosages in subjects of interest, e.g., humans. The therapeutically effective amount should be enough to exert a therapeutically useful effect in the absence of undesirable side effects in the subject to be treated with the composition.

Pharmaceutically acceptable carriers are well known to those skilled in the art and include, but are not limited to, from about 0.01 to about 0.1M and preferably 0.05M phosphate buffer or 0.8% saline. Such pharmaceutically acceptable carriers can be aqueous or non-aqueous solutions, suspensions and emulsions. Examples of non-aqueous solvents suitable for use in the presently disclosed subject matter include, but are not limited to, propylene glycol, polyethylene glycol, vegetable oils such as olive oil, and injectable organic esters such as ethyl oleate. Aqueous carriers suitable for use in the presently disclosed subject matter include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the presently disclosed subject matter can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the presently disclosed subject matter include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the presently disclosed subject matter include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Parenteral carriers suitable for use in the presently disclosed subject matter include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the presently disclosed subject matter can be mixed as needed with disintegrants, diluents, granulating agents, lubricants, binders and the like using conventional techniques known in the art. The carriers can also be sterilized using methods that do not deleteriously react with the compounds, as is generally known in the art. The compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Further, formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compounds can further be formulated for topical administration. Suitable topical formulations include one or more compounds in the form of a liquid, lotion, cream or gel. Topical administration can be accomplished by application directly on the treatment area. For example, such application can be accomplished by rubbing the formulation (such as a lotion or gel) onto the skin of the treatment area, or by spray application of a liquid formulation onto the treatment area.

In some formulations, bioimplant materials can be coated with the compounds so as to improve interaction between cells and the implant.

Formulations of the compounds can contain minor amounts of wetting or emulsifying agents, or pH buffering agents. The formulations comprising the compound can be a liquid solution, suspension, emulsion, tablet, pill, capsule, sustained release formulation, or powder.

The compounds can be formulated as a suppository, with traditional binders and carriers such as triglycerides.

Oral formulations can include standard carriers such as pharmaceutical grades of mannitol, lactose, starch, magnesium stearate, polyvinyl pyrollidone, sodium saccharine, cellulose, magnesium carbonate, etc.

In some embodiments, the pharmaceutical composition comprising the compound of the presently disclosed subject matter can include an agent which controls release of the compound, thereby providing a timed or sustained release compound.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-6

Study approval. All animal studies have been done in accordance with UAB and UNC Institutional Animal Care and Use Committee guidelines

Tumor implantation, treatment and harvest. For animal studies, 1*10⁵ B16F10 luciferase-tagged melanoma cells, 1*10⁶ GEMM6 melanoma cells or 2*10⁶ LCC cells were implanted subcutaneously into C57BL/6J mice (randomized, both males and females, 000664 The Jackson laboratory). When indicated, mice received intraperitoneal injections 7 days after implantation with 100 μl of 5 mg/kg cisplatin (Tocris), 100 μl of 10 mg/kg BVT948 (Tocris) or 100 μl of 30 mg/kg of vemurafenib. 5 mice were used per experimental group and tumors were harvested when they reached 1.5 cm in any direction.

Generation of transgenic mice. Lyz2-Cre:R26R^{tdTomato} mouse line were generated as previously described (36) MyD88-KO and Mer-KO mice were obtained from The Jackson Laboratory (008888 and 011122 respectively). Tyro3-KO and Axl-KO mice were obtained at Dr. Earp lab as previously described (36).

Immunohistochemistry analysis. Immunohistochemistry assay was carried out in formalin-fixed, paraffin-embedded 4 μm sections using primary antibodies against F4:80 (AbD Serotec), CD11b (Abcam), CD4 (eBioscience) or CD8a (eBioscience) on a Discovery Ultra Automated IHC staining system (Ventana). Sections were then imaged using a ScanScope XT microscope (Aperio) and quantified using Aperio software.

Peritoneal macrophage isolation and culture. Peritoneal macrophages were done as described previously(65) from 8-week-old C57BL/6J mice (randomized, both male and female). PBS (Gibco) was injected in the mural peritoneal cavity to release the macrophages. After recovering, cells were centrifuged at 1200 rpm for 5 minutes, resuspended in DMEM high glucose, 10% FBS, 1% penicillin-streptomycin (Gibco) and plated. 2 hours after the last step, cells were washed with PBS and maintained in DMEM/F12 supplemented with 10% FBS, 1% penicillin-streptomycin and 20 ng/ml of M-CSF (Prospec) for 2 days. Macrophages were treated for 24 hours with 50 ng/ml LPS (Sigma Millipore), 100 ng/ml IFNγ (BioLegend), 300 ng/ml PAM3CSK4 (InvivoGen), 107 cells/ml heat killed Listeria Monocytogenes (HKLM) (InvivoGen), 100 ng/ml Flagellin Salmonella typhimurium standard (InvivoGen), 10 ng/ml FSL1 (InvivoGen), 2.5 ug/ml ssRNA40 (InvivoGen), 2.5 ug/ml CL 075 (Tocris), 5 μM ODN1826, 50 μM Fludarabine (SelleckChem), 50 nM Momelotinib (SelleckChem), 5 μM BVT948 (Sigma Millipore), 200 μM PTP1b inhibitor III (Santa Cruz Biotechnology Inc.), 5 μM NSC87877 (Santa Cruz Biotechnology Inc.), 50 μM PTP1b inhibitor (Cayman), 1 mM PTP Inhibitor IV (Santa Cruz Biotechnology Inc.), 1 μM TSC401, 300 nM UNC2371 (SelleckChem), 300 nM BMS777607 (SelleckChem), 300 nM LDC1267 (SelleckChem) 1.5 μg/ml recombinant Pros1 (R&D systems), 200 ng/ml recombinant Gas6 (R&D systems) or 10-500 μM Cisplatin (Tocris). All components were added as described in the text. Cell culture. B16F10 (CRL-6475, ATCC), Lewis lung carcinoma (LLC) (CRL-1642, ATCC), KPC2713 (Yuliya Pylayeva-Gupta laboratory, UNC) and MDA-MB-231 (HTB-26, ATCC) were cultured in DMEM high glucose supplemented with 10% FBS, 1% penicillin-streptomycin (Gibco). GEMM6 (Dr. Alisha Holtzhausen, UNC), KPPC4662 (Dr. Yuliya Pylayeva-Gupta laboratory, UNC), KPPC4548 (Yuliya Pylayeva-Gupta laboratory, UNC) and E0771 (CRL-3461, ATCC) were maintained in RPMI 1640 supplemented with 10% PBS, 1% penicillin/streptomycin (Gibco) while PyMT cells were cultured in Ham's F-12K with 10% FBS, 1% penicillin-streptomycin (Gibco). Transwell assays were performed in 0.4 μm polyester membrane insert culture plates (Corning). 500,000 cells of each line were plated per well in DMEM/F12 supplemented with 10% FBS, 1% penicillin-streptomycin and 20 ng/ml of M-CSF for culture with mouse macrophages. SC cells (CRL-9855, ATCC) were cultured in IMDM with 10% FBS and 1% penicillin-streptomycin (Gibco). In transwell experiments with the SC cell line, cancer cells were also resuspended in IMDM media.

siRNA transfection assay. Non-targeting (Dharmacon), Pros1 (Dharmacon) and Gas6 (Dharmacon) siRNAs were transfected into B16F10 cells using Lipofectamine RNAiMAX reagent (ThermoFisher scientific) according to manufacturers' instructions, adding fresh DMEM high glucose media supplemented with 10% FBS and 1% penicillin-streptomycin (Gibco) 5 hours after the start of the experiment. 2 days after the start of transfection, B16F10 cells were lifted with 0.05% trypsin (Gibco) and used for transwell experiment

DAMP characterization assay. B16F10 or GEMM6 cells were resuspended in DMEM deficient media with 1% penicillin-streptomycin (Gibco) and incubated for 3 days. Conditioned media was collected, filtered with a 0.2 μm filter and incubated with 0.35 U/ml proteinase K-agarose beads (Sigma Millipore) or 40 U/ml of benzonase endonuclease (Sigma Millipore) for 4 hours at 37° C. under rotation. Conditioned media was two-fold diluted with DMEM/F12 with 10% FBS, 1% penicillin-streptomycin (Gibco) and 20 ng/ml of M-CSF (Prospec), supplemented with 5 μM BVT948 (Sigma Millipore) and added to primary mural macrophages for 24 hours.

RNA-seq. Total RNA was isolated with the RNeasy Plus Mini Kit (Quiagen). RNA quality was assayed using the NanoPhotometer spectrophotometer (Implen) and quantified using the RNA Nano 6000 Assay Kit of the Bioanalyzer 2100 system (Agilent Technologies, CA, USA). Sequencing libraries were generated using NEBNext Ultra™ RNA Library Prep Kit for the Illumina system (New England Biolabs) following manufacturer's recommendations. Library quality was checked on the Bioanalyzer 2100 system (Agilent). After clustering the index-coded samples, the library preparations were sequenced on the Ilumina HiSeq XTEN platform. Raw data were firstly processed through fastp software to eliminate adapter and poly-N and low-quality reads. Paired-end clean were aligned to the reference genome annotation files download from NCBI browser using the Spliced Transcripts Alignment to a Reference (STAR) software (Illumina). ClusterProfiler R package was used for statistical Reactome enrichment analysis.

qRT-PCR. RNA was isolated using the RNeasy Plus Mini Kit (Quiagen) and cDNA was prepared using the Reverse Transcription System (Promega) with one-hour reverse transcription process. qRT-PCR was done using the PowerUp™ SYBR™ Green Master Mix (Applied Biosystems) on a QuantStudio Pro Real-time PCR system (Applied Biosystems) with a 95° C. pretreatment of 10 min, 50 cycles of 95° C., 10 sec and 60° C., 1 min and melt curve at the end. Analysis was performed with the 2^{-ΔΔCT} method(136).

Coimmunoprecipitation and Western blot. Coimmunoprecipitation was performed using protease A/G agarose beads (Santa Cruz Biotechnology Inc.) and antibodies for PTP1b (Abcam) or STAT1 (Cell signaling). Samples were incubated for 3 days at 4° C. under rotation and washed three times with RIPA buffer before loading them in 10% Mini-PROTEAN TGX stain-free gels (Biorad). After running the electrophoresis in the Mini-PROTEAN Tetra System (Bio-Rad) proteins were transferred to a 0.2-μm nitrocellulose Trans-Blot Mini Turbo Transfer Pack (Bio-Rad) using a Trans-Blot Turbo Transfer System (Bio-Rad). Membranes were blocked with 5% BSA (Fisher scientific) for 1 hour and blotted with antibodies against MyD88 (Cell Signaling), total STAT1 (Cell Signaling), tyrosine 701 phosphorylated STAT1 (Cell signaling), serine 727 phosphorylated STAT1 (Cell signaling), PTP1 b (Abcam) and Mer (Thermo Fisher scientific). Detection was performed with the ECL HPR-linked anti-rabbit antibody (Cell signaling) and the Clarity-Max Western ECL substrate (Bio-Rad). Blots were imaging with ChemiDoc MP Imaging System (Bio-Rad) and quantitated using ImageJ software (NIH).

Immunofluorescence staining and quantitation. After treatments, mouse macrophages were fixed with 4% formaldehyde (Electron Microscopy Sciences) and permeabilized with acetone (Fisher scientific). Samples were blocked using BlockAid blocking solution (Thermofisher scientific), were incubated with primary antibodies to MyD88 (Cell signaling), CD86 (Biolegend) and total STAT1 (Cell signaling) and Alexa Fluor 488-labeled secondary antibody (LifeTech) and were mounted with SlowFade Gold Antifade Mountant with DAPI (Invitrogen). Images were taken with using a BZ-X810 all-in-one fluorescence microscope (Keyence). Fluorescence intensity was measured with BZ-X800 software while colocalization was analyzed using ImageJ software with JACoP plugin (NIH)

MyD88/STAT1 expression in human tumors. Analysis of MyD88 and STAT1 expression and association in human tumors were performed using The Gene Expression Profiling Interactive Analysis tools (http://qepia.cancer-pku.cn)

ChIP assay. ChIP was performed as previously described with modifications(137, 138). Briefly, 3-5 million cells were PBS-washed and crosslinked with formaldehyde (1%), followed by quenching with 0.125 M glycine. After washing with cold PBS, cells were suspended in lysis buffer (50 mM HEPES pH 7.5, 140 mM NaCl, 1 mM EDTA, 10% glycerol, 0.5% NP-40, 0.25% TritonX-100, and protease inhibitors) and subjected to sonication to shear the chromatin DNA into sizes ranging from 200 to 500 bp. The fragmented chromatin was centrifuged at 15,000 rpm for 15 min at 4° C. The supernatant was incubated with STAT1 antibody (Cell signaling) and Protein G Dynabeads magnetic beads (Invitrogen) at 4° C. overnight. After six times wash with RIPA wash buffer (50 mM Hepes-KOH, pH 7.6, 500 mM LiCl, 1 mM EDTA, 1% NP-40, and 0.7% Na-Deoxycholate) and once wash with TE buffer (10 mM Tris-HCl pH 8.0, 1 mM EDTA, and 50 mM NaCl), chromatin was eluted from beads in elution buffer (50 mM Tris-HCl pH 8.0, 10 mM EDTA, and 1% SDS). After reverse crosslinking, ChIP DNA was purified using DNA purification columns (Zymo Research) and subjected to downstream quantitative real-time PCR analysis (ChIP-qPCR).

Statistical analysis. All statistical analysis were detailed in figure legend and performed using GraphPad Prism software (GraphPad Software Inc.). A p value less than 0.05 was considered as statistically significant. Graphs display the mean value±SEM per experimental group. The number of studies done to arrive at the total sample size (n) is detailed in each figure legend. When not explicitly stayed, one experiment with the indicated sample size was performed to get the results

### Example 1

### Tumor Secretions Suppression of Macrophage MyD88, a Key TLR Signaling Associated Adapter Protein.

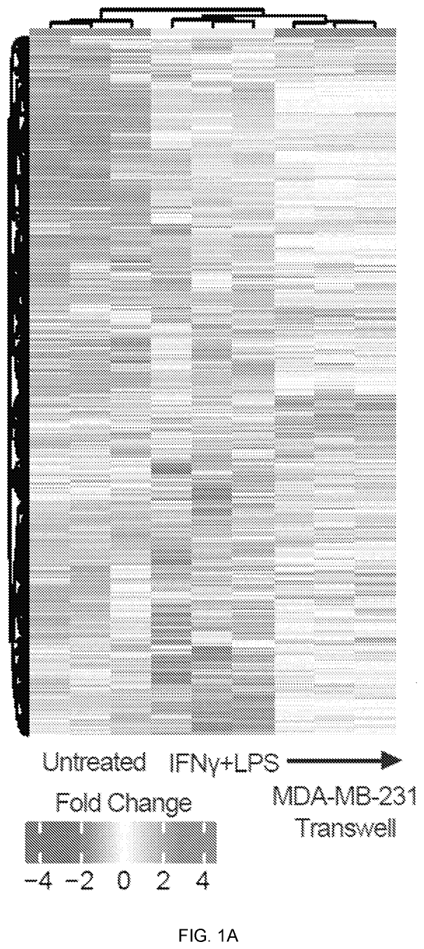

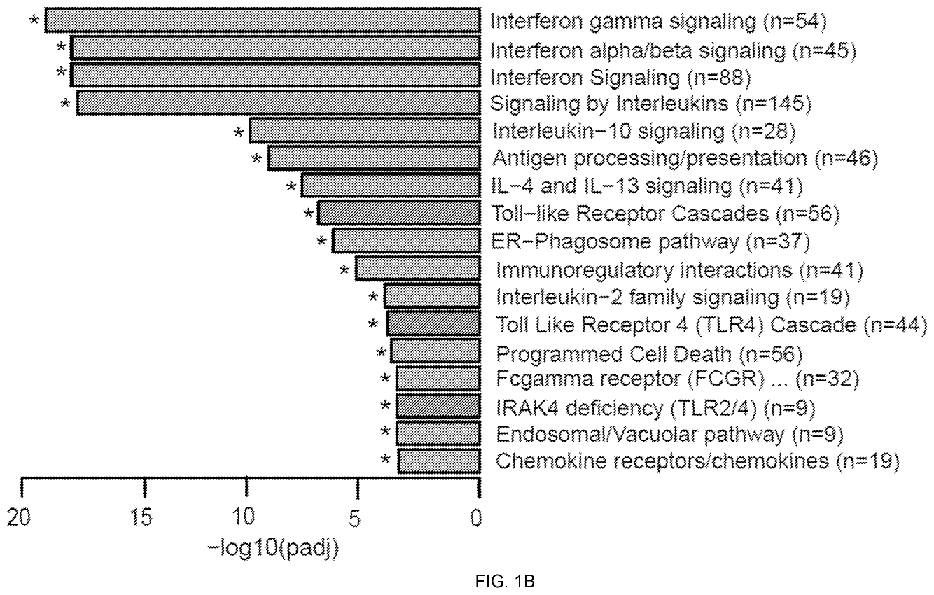

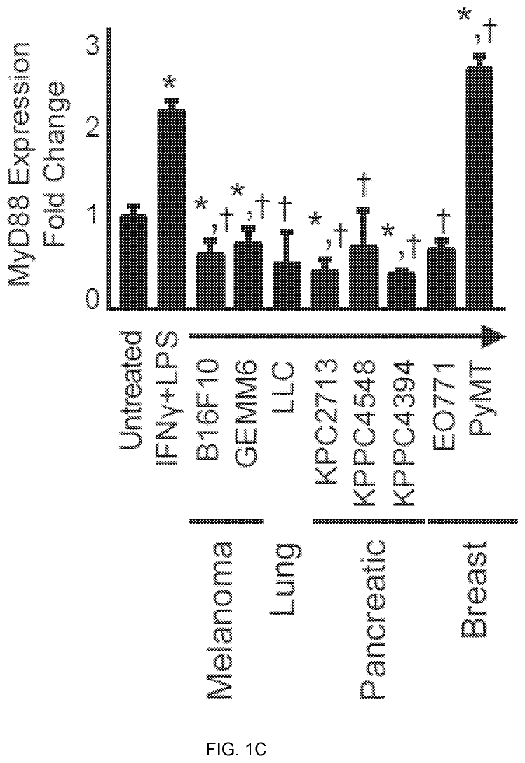

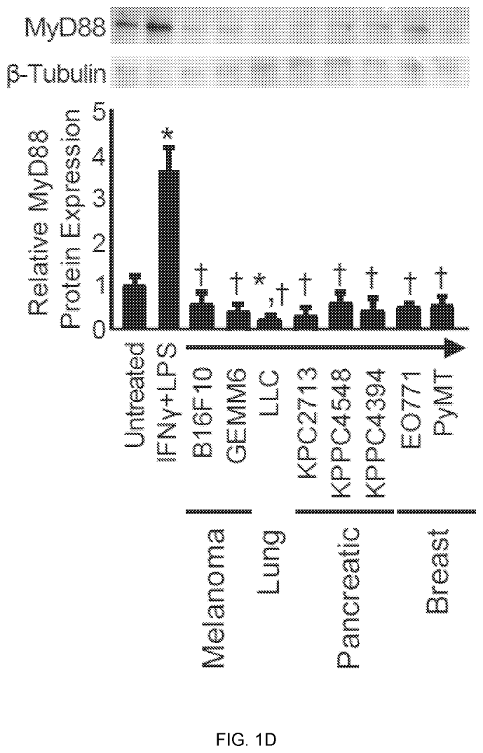

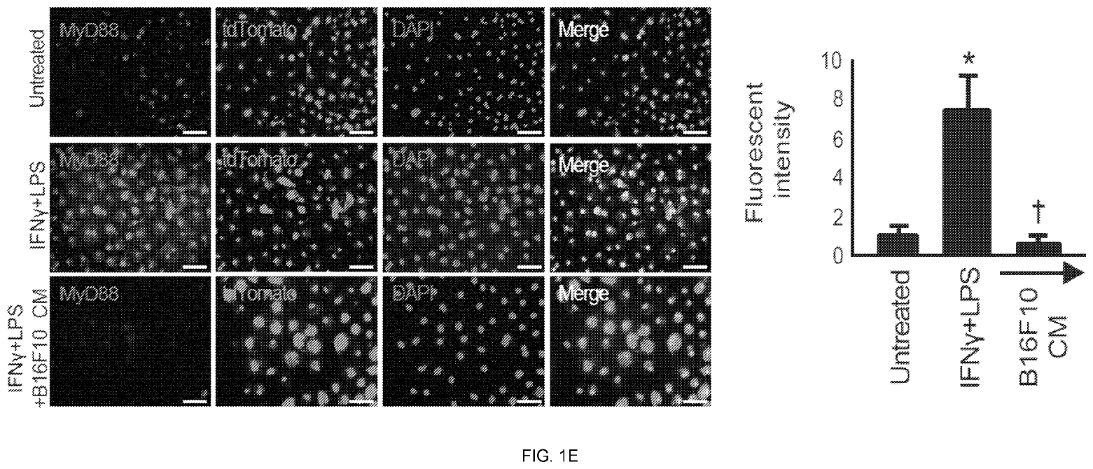

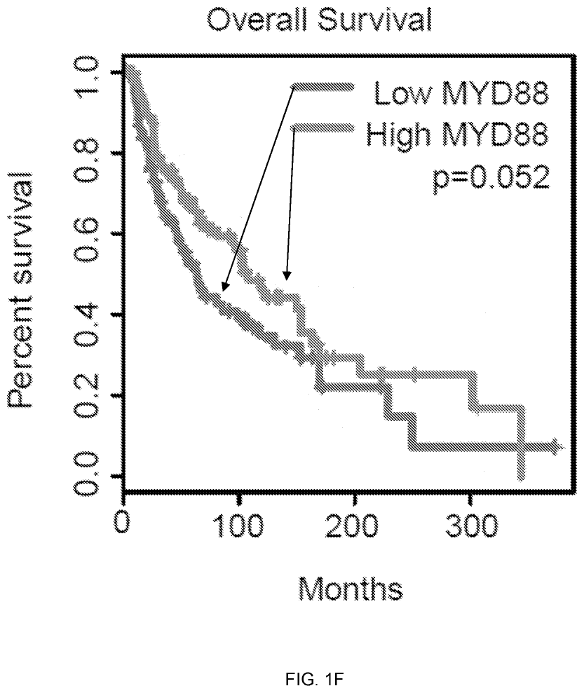

Tumors express several well characterized contact-mediated suppressors of the macrophage pro-inflammatory (M1) response, including CD24 and CD47. However, the effects of tumor secretions on the macrophage M1 response are less clear. Utilizing a transwell assay in which macrophages are co-cultured with tumor cells, though physically separated by a 0.4 μm pore size membrane (37), the effects of tumor secretions on the macrophage pro-inflammatory response were assayed. In this system, untreated human macrophages (SC cell line(38-40)) or those stimulated with 100 ng/ml interferon gamma (IFNγ) and 50 ng/ml lipopolysaccharide (LPS) were cultured in the presence or absence of the human breast cancer cell line MDA-MB-231(41-43). After 24 hours of co-culture, macrophage mRNA was isolated and analyzed using RNAseq to determine, in an unbiased manner, which pathways are affected by tumor secretions. While many genes associated with protein synthesis were upregulated in the presence of tumor cells, several pathways associated with the anti-tumor response were downregulated, including interferon (IFN) and interleukin (IL) signaling (FIG. 1B; “Signaling by Interleukins”). Interestingly, it was discovered that three different Toll-like receptor (TLR) associated pathways are also suppressed in the presence to tumor secretions (FIG. 11B; “Toll-like Receptor Cascades”, “Toll Like Receptor 4 (TLR4) Cascade”, and “IRAK4 deficiency”).

In the context of cancer, the release of DAMPs from dying tumor cells is thought to be an initiating event for the inflammatory response(44-46). Whether due to hypoxia or nutrient deprivation, which are frequent during cancer progression(47-49), as tumor cells undergo cell death, proteins and nucleic acids are released. These DAMPs should then activate the TLR family of receptors on macrophages, causing a signaling cascade resulting in expression of pro-inflammatory genes(50-53). A key signaling transducer common to all but one member of the TLR family is the adapter protein MyD88(54). Demonstrating the importance of MyD88 in the inflammatory response, mice lacking MyD88 are severely immunocompromised because of their inability to recognize, and respond to, bacterial, viral and fungal pathogens (55-57).

Based on the importance of MyD88, it was hypothesized that if tumor cells can suppress macrophage expression of MyD88, they may suppress the pro-inflammatory DAMP response. Therefore, the RNAseq data was analyzed, which was then confirmed by qRT-PCR following a transwell assay and found that while M1 induced macrophages expression of MyD88 is increased, co-culture with tumor cells led to a 25% reduction. To determine whether suppression of macrophage MyD88 is a conserved mechanism of tumor-derived innate immune inhibition, assays were conducted to determine whether a panel of murine tumor cell lines could similarly suppress MyD88 mRNA levels. Using the disclosed transwell assay, melanoma (B16F10(58, 59), GEMM6(60, 61)), lung (LLC(62, 63)), pancreatic (KPC2713, KPPC4548, KPPC4394(36)) and breast (E0771(64, 65), PyMT(66, 67)) cancer cells were co-cultured with M1-induced primary macrophages and the changes were analyzed on MyD88 mRNA expression (FIG. 1C). It was found that in the presence of IFNγ and LPS, MyD88 expression of murine C57BL/6J peritoneal macrophages was increased by 2.13-fold (+/−SEM). However, when co-cultured with tumor cells, MyD88 mRNA levels were reduced to naïve levels or below, with the exception of PyMT (FIG. 1C). To corroborate these findings, Western blot analysis was utilized and it was found that macrophage MyD88 protein levels, which are increased by IFNγ and LPS stimulation, were similarly suppressed to naïve levels or below by all tumor lines tested (FIG. 1D). While it is unclear why PyMT cells did not reduce macrophage MyD88 mRNA levels but did reduce associated protein levels, it may be due to alterations in mRNA half-life, processing or translation that cause dissociation of the two measures. To further corroborate these surprising findings, immuno-fluorescent staining of genetically labeled peritoneal macrophages isolated from Lyz2-Cre(68):lox-stop-lox tdTomato(69) mice was used to determine the effect of B16F10 conditioned medium on MyD88 localization in M1 macrophages. After culturing unstimulated or IFNγ and LPS induced macrophages in the presence or absence of B16F10 conditioned medium for 24 hours, confocal microscopy was used to monitor expression and localization of MyD88 (FIG. 1E). In line with the Western blot analysis findings (FIG. 1D), macrophage MyD88 levels increased upon IFNγ and LPS stimulation, but were globally reduced in the presence of B16F10 conditioned medium. Spectral analysis was used to quantitate relative MyD88 protein levels, which revealed that fluorescence was increased 7.43-fold (+/−SEM) upon IFNγ and LPS stimulation but was reduced to naïve levels by B16F10 condition medium, further demonstrating that tumor secretions are responsible for the reduction of macrophage MyD88 levels. Taken together, these surprising findings show that not only is MyD88 suppression a conserved mechanism in mice and humans, it is also characteristic of diverse tumors as a mechanism of innate immune suppression.

Because MyD88 is essential for most TLR signaling(70, 71) and is expected to be an important factor in innate immune activation, Gepia was utilized to analyze TCGA data to determine whether MyD88 expression is associated with melanoma patient survival. This analysis shows that patients with reduced MyD88 expression tended to have worse outcomes (FIG. 1F).

### Example 2

Tumor Suppression of MyD88 is Associated with Reduced Macrophage Response to TLR Agonism.

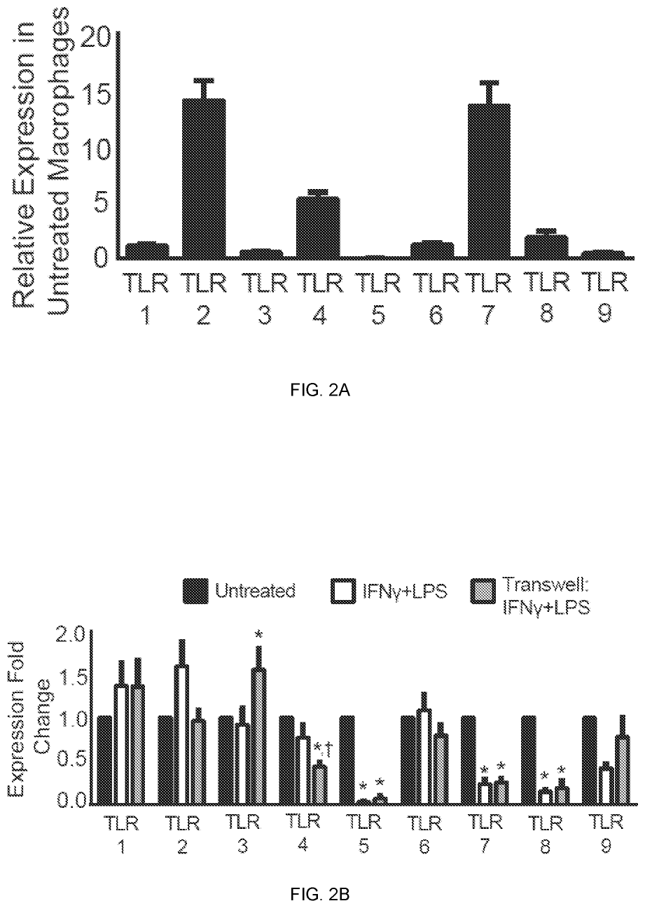

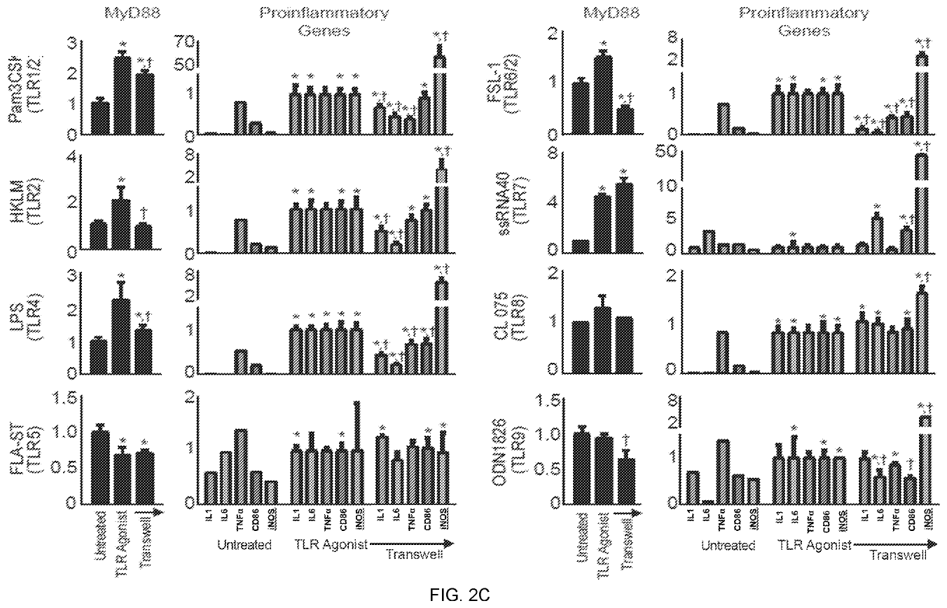

The members of the TLR family, with the exception of TLR3, are dependent upon MyD88 for downstream pro-inflammatory signaling (22, 46, 54-56, 70-72). Herein it was hypothesized that tumor-mediated reduction in MyD88 expression would lead to decreased TLR response across the spectrum of TLR agonists. However, before determining whether TLR agonism was affected, the expression of TLR receptors on naïve peritoneal macrophages was first characterized by qRT-PCR. It was observed that TLR2, TLR4 and TLR7 are the most highly transcribed, though all the receptors are expressed at some level (FIG. 2A). It was next determined whether M1 induction or the presence to tumor secretions affected TLR levels. To do so, RNA was isolated from untreated or IFNγ and LPS induced peritoneal macrophages in the presence or absence of transwell co-cultured B16F10 cells, and qRT-PCR was performed. Apart from TLR5, TLR7, TLR8 and TLR9, which were reduced during IFNγ and LPS stimulation, expression levels of other receptors were not significantly altered. As compared to M1 macrophages, only TLR4 showed significantly reduced expression in the presence of B16F10 cells while TLR3 was increased (FIG. 2B), indicating that, for the most part, tumor cells do not suppress expression of individual receptors.

After determining that TLR expression was not substantially affected by tumor secretions, it was next determined whether tumor secretions affected MyD88 or pro-inflammatory gene expression upon TLR agonism. It was found that individual TLR agonism of TLR1/2 (Pam3CSK4), TLR2 (HKLM), TLR4 (LPS), TLR6/2 (FSL-1) and TLR7 (ssRNA40) led to the upregulation of MyD88 (FIG. 2C). As shown by FIG. 2C, in the cases of TLR1/2, TLR2, TLR4, and TLR6/2 agonism, tumor suppressed MyD88 was associated with reduced pro-inflammatory gene expression in the presence of tumor cell secretions. In the cases of TLR5 (FLA-ST) or TLR8 (CL075) agonism, when macrophage MyD88 expression was not significantly affected by transwell co-culture with B16F10 cells, pro-inflammatory gene expression was similarly unaffected (FIG. 2C). Alternatively, in the lone case where TLR agonism increased MyD88 expression in the presence of tumor cells (TLR7, ssRNA40), there was increased pro-inflammatory gene expression. In all cases, there was a direct correlation between MyD88 expression and the expression of pro-inflammatory genes (IL-1, IL-6, TNFα and CD86). With the exception of TLR5 agonism, it was also observed that transwell co-culture induced macrophage iNOS expression (FIG. 2C). In peritoneal macrophages, iNOS is regulated by Stat3 activation. As tumor cells are known to induce Stat3 activation in macrophages(73-79), upregulation of iNOS may be a tumor-mediated suppressor of the adaptive immune response, as iNOS can deactivate T cells(80-85).

Taken together, these results demonstrate that tumor secretions have the wide-ranging ability to suppress downstream activation of most TLR receptors. As TLR agonists are currently in development and clinical trials (reviewed in(86, 87)), these findings show that tumor-derived suppression of TLR downstream signaling limit the efficacy of TLR agonists in patients.

### Example 3

### Tumor-Mediated Decreases in Stat1 Activation Cause Reduction in MyD88 Expression.

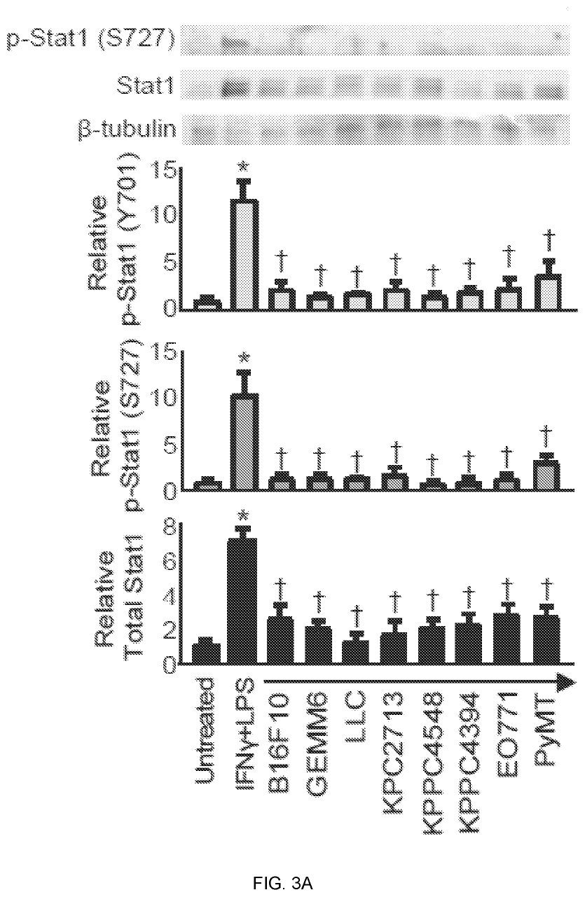

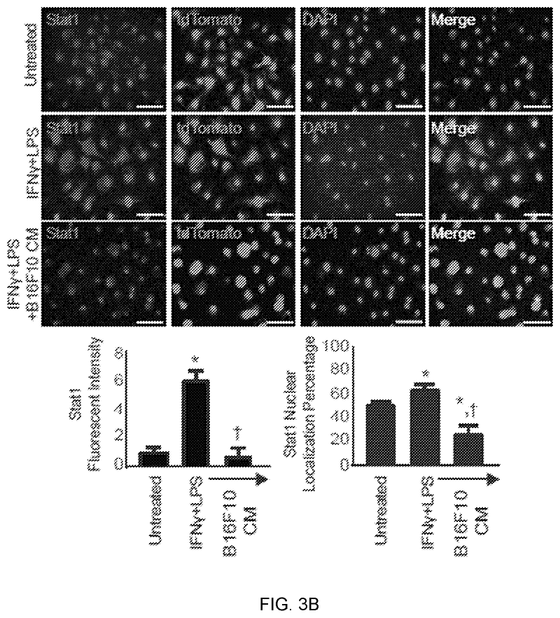

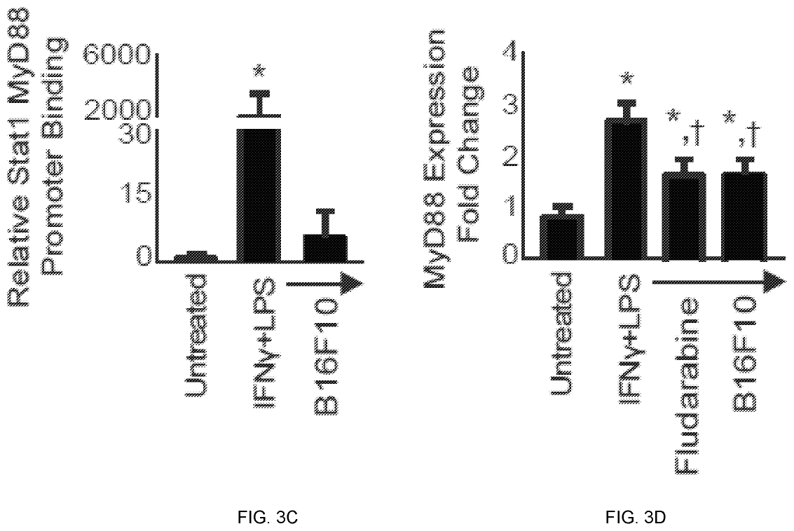

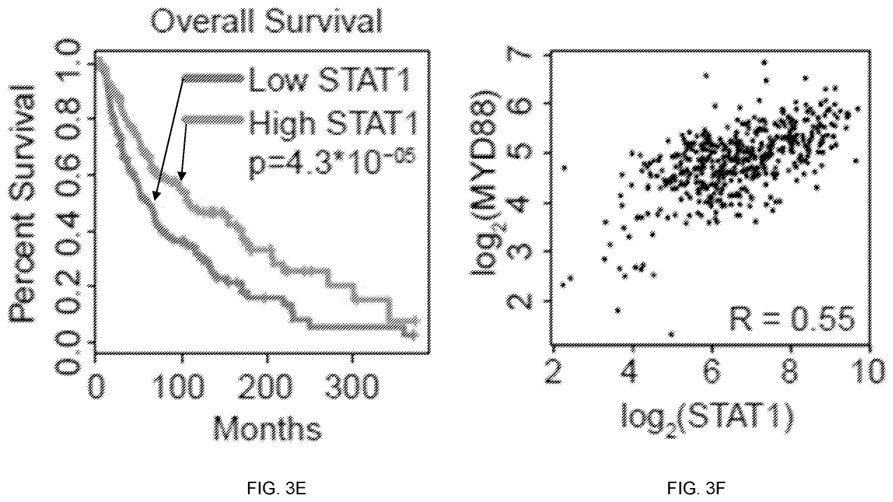

After establishing that tumors suppress expression of the key TLR adapter protein MyD88, other regulatory signaling pathways that could potentiate the effect were reviewed and considered. It was hypothesized herein that while M1 stimulation induces Stat1 activation, tumor secretions may effectively reduce Stat1 activity, thereby eliciting the downregulation of MyD88 in pro-inflammatory macrophages. To test this hypothesis, Western blot analysis of untreated or M1 induced macrophages in the presence or absence of our tumor cell line panel was utilized. As expected, IFNγ and LPS stimulation increased total Stat1 by 7-fold as well as phosphorylation at residues Tyrosine 701 and Serine 727 by 10-fold (FIG. 3A). However, in all cases, co-culture of pro-inflammatory macrophages with tumors of diverse origins reduced macrophage total and phospho-Stat1 levels to naïve levels (FIG. 3A). As increased Stat1 phosphorylation is associated with nuclear translocation and transcription factor activity, immunofluorescent staining was utilized to determine the effects of tumor conditioned medium on Lyz2-Cre:tdTomato labeled macrophage Stat1 localization. An increase in total and nuclear Stat1 was observed when macrophages were treated with IFNg and LPS. However, when B16F10 conditioned medium was added to pro-inflammatory macrophages total Stat1 was reduced to naïve levels (FIG. 3B), corroborating the Western blot analysis findings (FIG. 3A). Nuclear localization of Stat1 was increased by 14% (+/−SEM) upon M1 activation but was reduced by roughly 40% upon culture with B16F10 conditioned medium (FIG. 3B).

In silico analysis of MyD88 promoter elements identified potential Stat1 binding sites. Using chromatin immunoprecipitation (ChIP), the presence of Stat1 was determined at proximal and distal MyD88 promoter sites. While Stat1 association at the distal site was not detected (data not shown), M1 induced macrophages have increased association of Stat1 at the proximal site while transwell co-culture reduced binding to naïve levels (FIG. 3C). This supports the idea that tumor reduction of pro-inflammatory macrophage Stat1 levels leads to reduced Stat1 nuclear translocation and transcription factor activity.

To confirm that Stat1 activation is essential for MyD88 expression, it was determined whether the Stat1 inhibitor, Fludarabine, would reduce MyD88 expression in pro-inflammatory macrophages. To do so, pro-inflammatory macrophages were treated with 50 μM Fludarabine(95, 96), which reduced Stat1 expression levels. For comparison, we transwell co-cultured M1 macrophages with B16F10 tumor cells, observing comparable reduction of MyD88 expression by Stat1 inhibition and transwell co-culture (FIG. 3D). Canonical Stat1 signaling relies on Jak1/2 as an upstream activator. It was therefore determined whether Jak1/2 inhibition, using the clinical compound Momelotinib, would have similar effects in reducing MyD88 expression. Momelotinib treatment, however, did not significantly alter macrophage Stat1 expression or MyD88 expression implying that changes in Stat1 activation are not regulated upstream by the canonical pathway.

Because reduced MyD88 expression is associated with worse outcomes in melanoma patients (FIG. 1F), and because Stat1 regulates MyD88 expression in our murine model, using the TCGA database it was determined whether reduced Stat1 expression would also be associated with worse patient outcomes. For melanoma patients, reduced Stat1 expression was associated with worse outcomes (FIG. 3E). Further, there is a strong positive correlation (R=0.55) between Stat1 expression and MyD88 expression in melanoma patients (FIG. 3G), reinforcing the idea that suppression of Stat1-mediated MyD88 expression leads to worse patient outcomes.

### Example 4

Tumor-secreted Pros1 suppresses MyD88 expression via the macrophage Mer:PTP1b axis.

These results demonstrate that tumor secretions reduce MyD88 expression in pro-inflammatory macrophages through a Stat1 dependent mechanism. Moreover, the canonical Jak:Stat1 pathway does not seem to play a role, leading us to inquire through which molecular mechanisms tumor secretions modulate Stat1 activity.

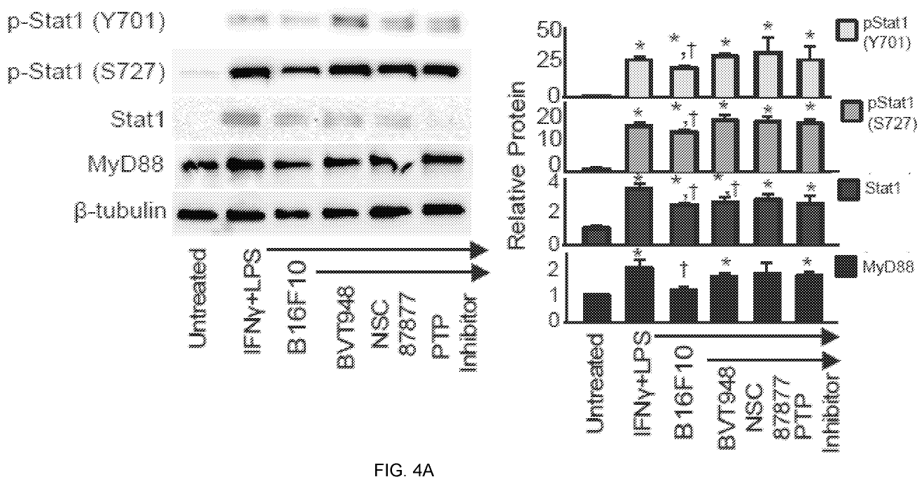

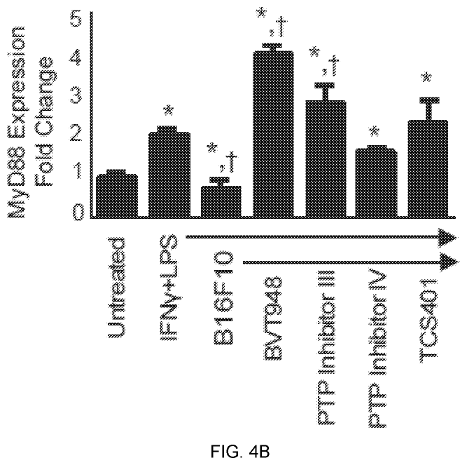

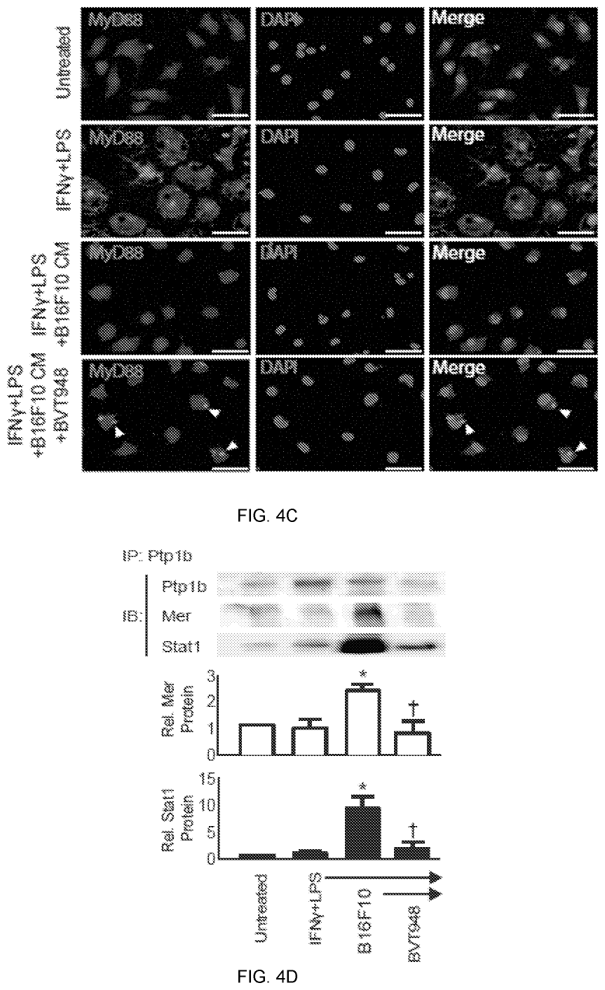

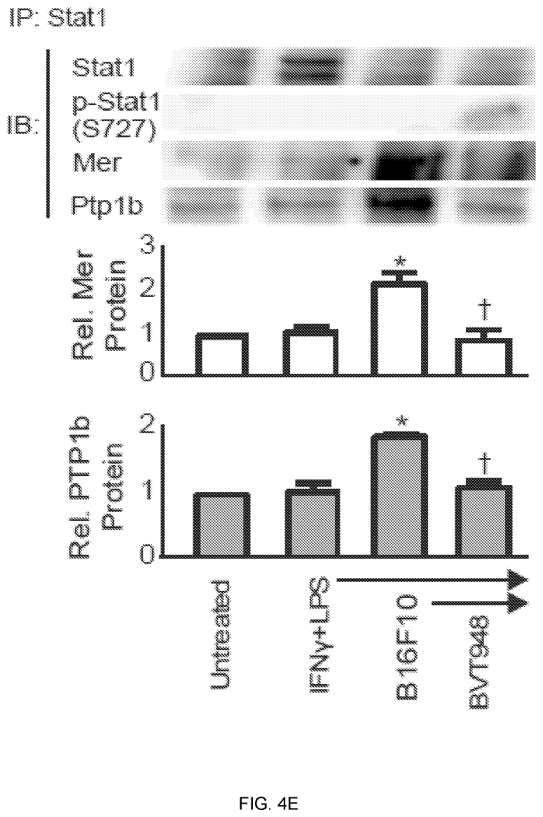

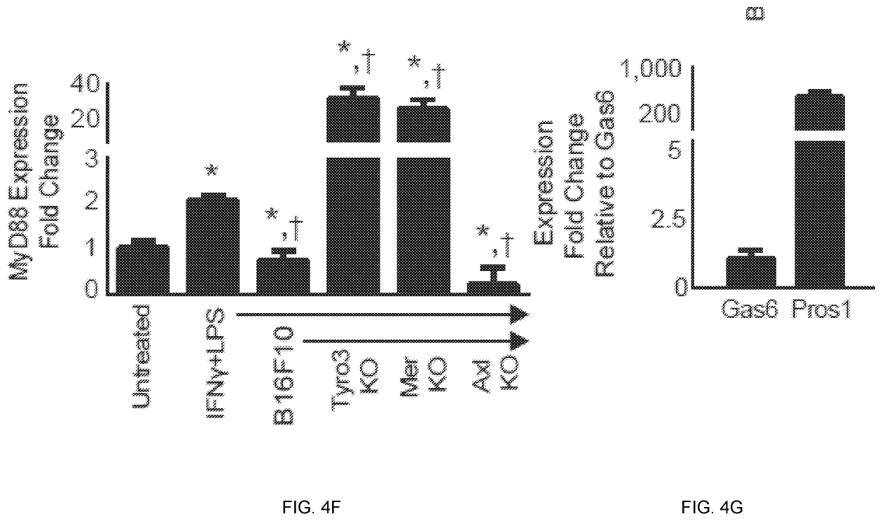

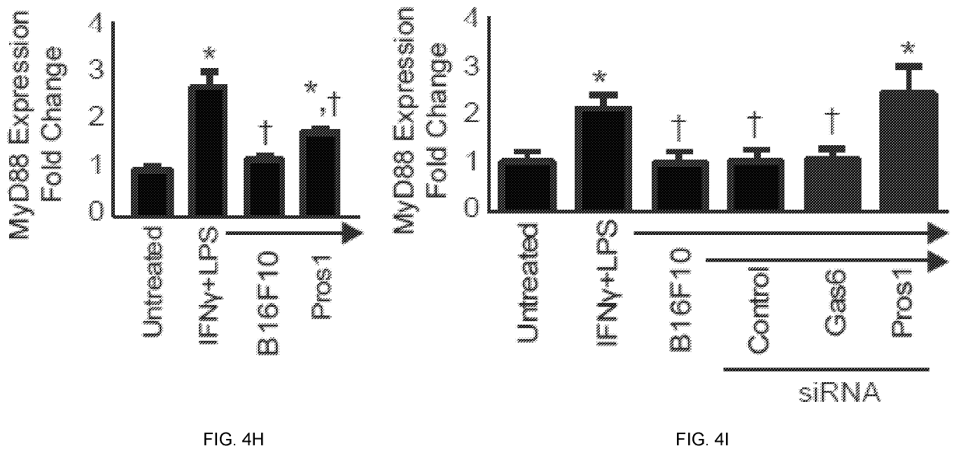

Others have shown that Stat1 activity is effectively suppressed by the protein tyrosine phosphatase (PTP) family of proteins (97-99). It was therefore decided to assay what effect pharmacological PTP1 b inhibition would have on Stat1 and MyD88 in the disclosed model. Using Western blot analysis, total and phosho-Stat1 was assayed, as well as MyD88, levels in naïve and M1 induced macrophages, in the presence or absence of tumor cells and with different PTP1 b inhibitors. As we found previously, tumor secretions suppressed both MyD88 and Stat1 levels. However, in the presence of three different PTP1 b inhibitors (BVT948, NSC87877, PTP inhibitor) Stat1 phosphorylation at Tyrosine 701 and Serine 727 were increased (FIG. 4A). PTP1b inhibition in our transwell assay also restored MyD88 protein and mRNA levels (FIGS. 4A and 4B). Because PTP1b inhibition restored MyD88 expression in pro-inflammatory macrophages in the presence of tumor secretions, we used immunofluorescent imaging to determine changes in MyD88 subcellular localization. When M1 macrophages were cultured in B16F10 conditioned medium, overall MyD88 protein levels were reduced as shown in FIG. 1E, but treatment with BVT948 led to increased MyD88, though primarily in puncta in the perinuclear space, probably in the early stages of translation in the endoplasmic reticulum (FIG. 4C). PTP1b inhibition was also associated with rescued expression of pro-inflammatory markers, like CD86.

PTP1 b forms ternary complexes with the immune suppressive receptor tyrosine kinase Mer. It was therefore hypothesized that Mer activation may increase ternary complex formation between Mer, PTP1b and Stat1, leading to PTP1b-mediated dephosphorylation of Stat1. To test this hypothesis, co-immunoprecipitation of PTP1b was performed. It was observed that in the presence of B16F10 cells, the association of PTP1b with Mer and Stat1 was increased. However, addition of the PTP1b inhibitor, BVT948, reduced the association of PTP1 b with both Mer and Stat1 (FIG. 4D). This finding was confirmed by co-immunoprecipitating Stat1 from untreated and M1 macrophages in the presence or absence of tumor cells with or without PTP1b inhibition (BVT948). These findings were corroborated, showing that PTP1 b inhibition led to reduced ternary complex formation, but also to restoration of Stat1 S727 phosphorylation (FIG. 4E). Taken together, these results demonstrate that upon co-culture, association of macrophage Mer with PTP1 b and Stat1 is increased. With increased association, PTP1 b activity results in the dephosphorylation of Stat1 and reduces nuclear translocation and MyD88 promoter binding, reducing MyD88 expression. PTP1b inhibition prevents ternary complex formation, rescuing Stat1 phosphorylation and downstream transcription factor activity.

To confirm and extend these observations, it was determined whether other members of the Tyro3/Axl/Mer (TAM) family were involved in MyD88 suppression. To do so, macrophages from Tyro3(100-104) Mer(74, 105-110) and Axl(111-116) knockout mice were isolated, induced the M1 phenotype and transwell co-cultured them with B16F10 cells. While MyD88 expression in macrophages from wildtype or Axl knockout mice were suppressed by tumor cells, neither Tyro3 nor Mer knockout macrophages could be suppressed (FIG. 4F). It should be noted that M1 stimulation of all three KO macrophages led to equivalent expression of MyD88, making it highly unlikely that MyD88 expression levels of Mer and Tyro3 KO macrophages are because of exuberant pro-inflammatory activation. Together, these findings indicate that both Mer and Tyro3, but not Axl, play a role in suppression of MyD88 expression. Therefore, co-immunoprecipitation was performed to determine whether Mer and Tyro3 form heterodimers to facilitate MyD88 suppression. Heterodimer formation was not observed (data not shown) though it cannot be ruled out the possibility of Mer and Tyro3 interaction. Mer suppressive activity is often associated with its kinase activity. Therefore, transwell co-cultured pro-inflammatory macrophages were treated with Mer kinase inhibitors, though none rescued MyD88 expression, suggesting a Mer kinase independent function.

There are five known ligands for the TAM family, including Gas6 (117-119), Pros1(36, 101, 120), Tub, and Tulp1(105, 121, 122), and Gal3 (100, 123, 124). Of the ligands, Gas6 and Pros1 have been studied in the most detail so their expression was assayed in B16F10 cells. Using qRT-PCR, it was found that Pros1 expression is 500-fold that of Gas6 in naïve B16F10 cells (FIG. 4G). Knowing that B16F10 cells express both Gas6 and Pros1, a gain-of-function approach was utilized in which the effects of exogenous ligand addition were compared to transwell co-cultured cells to determine the roles of individual ligands on macrophage MyD88 expression. When added at 1.5 ug/ml Pros1, which has previously been shown equivalent to what is secreted by IFNγ-treated B16F10 cells, it was observed that MyD88 expression was reduced comparably to that of tumor cell secretions (FIG. 4H) while 200 ng/ml Gas6 had limited effect. A loss-of-function approach was also used, utilizing siRNA to knock-down either Gas6 or Pros1 mRNA levels in tumor cells prior to co-culture with pro-inflammatory macrophages. Knock-down efficiency of Pros1 and Gas6 exceeded 90% for each target. Neither control nor Gas6 siRNA treatment of tumor cells reduced suppression of macrophage MyD88 expression levels (FIG. 41). However, Pros1 siRNA treatment of tumor cells prior to co-culture prevented them from suppressing macrophage MyD88 expression (FIG. 41). Together, this gain- and loss-of-function approaches demonstrate that tumor-secreted Pros1 suppresses macrophage MyD88.

### Example 5

### PTP1b Inhibition Rescues Tumor DAMP TLR Activation

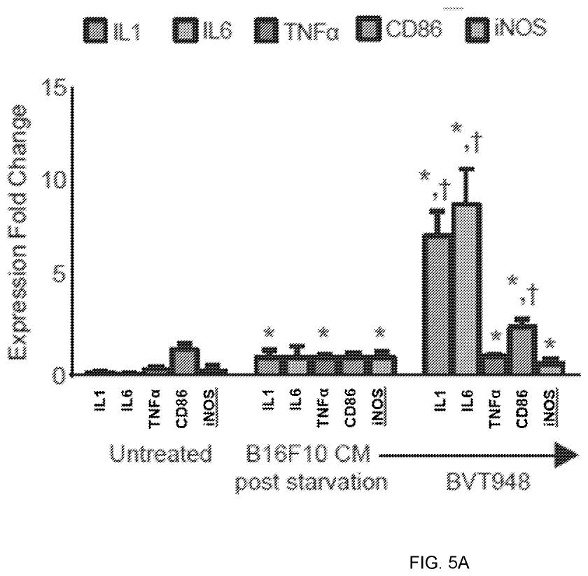

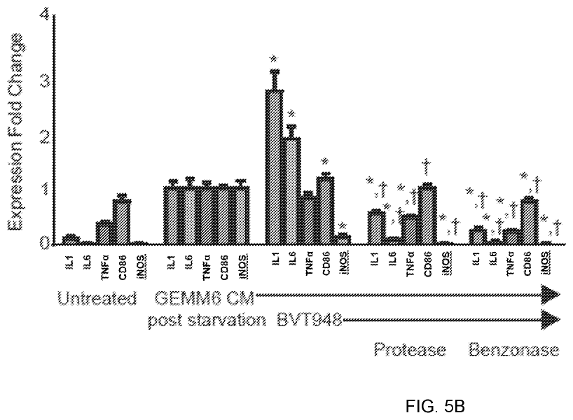

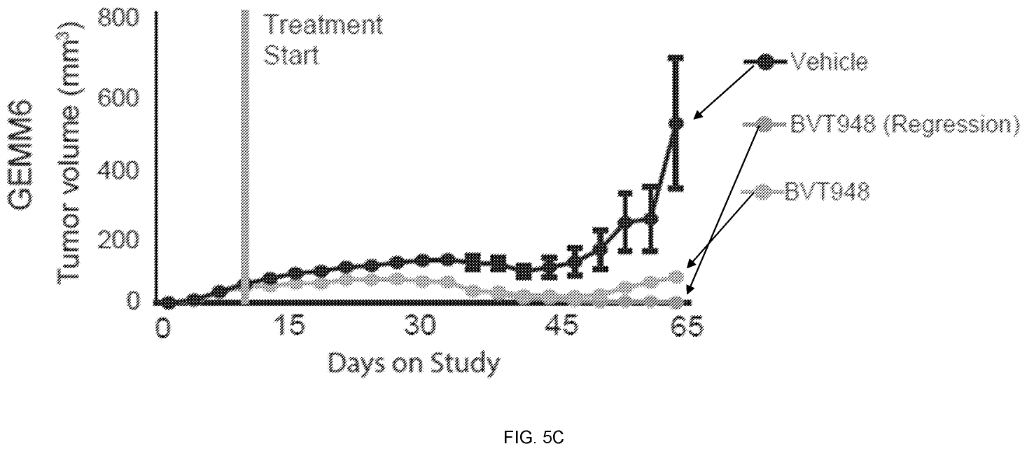

DAMPs are released by dead or dying cells and activate the spectrum of TLRs to initiate the macrophage pro-inflammatory response (125-129). In the context of cancer, because of limited blood flow, tumors can undergo nutrient deprivation and/or hypoxia which may lead to DAMP release. To model this scenario, we deprived B16F10 cells of nutrients by culturing the cells without fetal bovine serum (FBS) and essential amino acids for 3 days, thereby inducing necrotic and apoptotic cell death. After deprivation, conditioned medium was passed through a 0.2 μm filter to remove cell debris, though tumor secreted DAMPs are still present in the conditioned medium. B16F10 DAMP containing medium was applied to macrophages, resulting in relatively minor activation of pro-inflammatory gene expression, as compared to unstimulated naïve macrophage controls (FIG. 5A). Because tumor-secreted Pros1 remains in the medium, macrophages were co-treated with BVT948 and were able to restore DAMP mediated macrophage activation (FIG. 5A). Because DAMPs are a near-universal feature of cells and because many tumors undergo nutrient deprivation, a similar assay was performed on GEMM6 melanoma cells, which has also shown to reduce macrophage MyD88 expression (FIGS. 1C, 1D). Much like B16F10 cells, 0.2 μm filtered medium from nutrient deprived GEMM6 cells led to a modest increase in macrophage M1 response, as compared to unstimulated naïve macrophages. Again, addition of the PTP1b inhibitor BVT948 rescued pro-inflammatory gene expression (FIG. 5B). To identify which form of DAMPs, proteins or nucleic acids, have the most effect on the pro-inflammatory response, we treated GEMM6 nutrient deprivation conditioned medium with protease or benzonase to remove protein or nucleic acids, respectively. Even in the presence of BVT948, elimination of DAMP proteins or nucleic acids significantly reduced macrophage activation, though benzonase treatment had the most profound effect (FIG. 5E), underscoring the importance of tumor released DAMPs in macrophage activation.

Since PTP1 b inhibition is effectively able to unmask tumor DAMPs to improve the macrophage pro-inflammatory response, it was next assayed whether BVT948 treatment would improve outcomes in a preclinical melanoma model. GEMM6 melanoma bears the BrafV600E mutation with Pten loss, which is the most common mutation burden in human melanoma (60, 130). After orthotopic implantation in syngeneic C57BL/6 mice, treatment began when tumors were small, but palpable (50 mm³). Of the 8 mice per treatment arm, all but one vehicle treated tumor bearing mouse progressed. However, 6/8 BVT948 treated mice showed reductions in tumor growth with 3/8 completely regressing and immune to rechallenge 1 month later. The other 2/8 mice from the study arm died, mostly likely due to drug toxicity issues or autoimmune response (FIG. 5C).

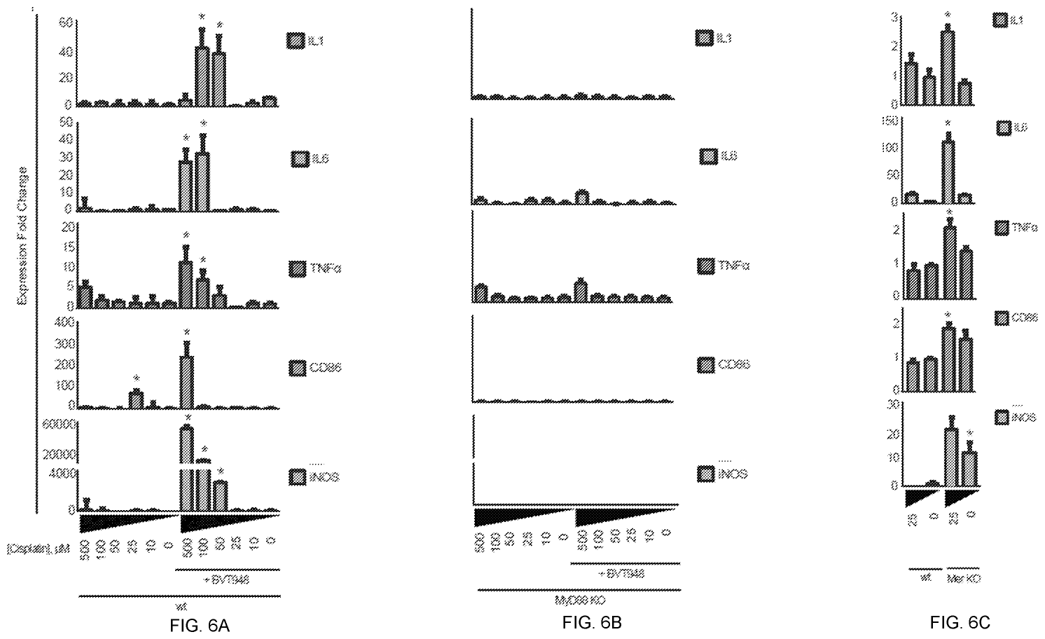

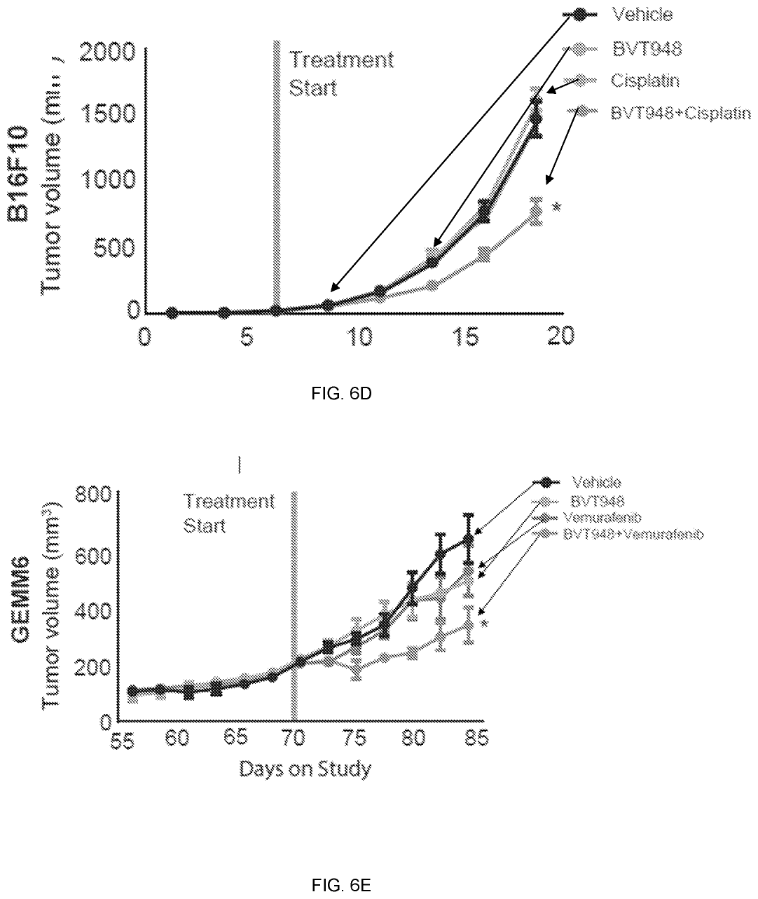

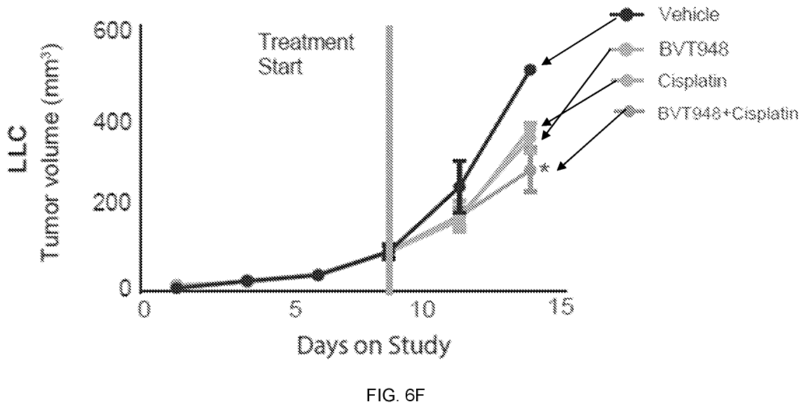

After the initial success, it was hypothesized that most, if not all, tumors are likely to release DAMPs during cancer progression. Therefore, it was next tested whether BVT948 as a monotherapy, would improve outcomes in B16F10, LLC or larger GEMM6 tumors. For aggressive B16F10 and LLC tumors, treatment began when tumors were 50 mm³, 7-9 days after implantation, and GEMM6 tumors when they reached 200 mm³. BVT948 monotherapy did not significantly reduce B16F10 or GEMM6 tumor growth as compared to vehicle treated controls (FIGS. 6D,6F) though LLC was reduced by 30% (FIG. 6E). BVT948 monotherapy affected the intra-tumoral immune infiltrate of each tumor differently, with some increases in macrophage and T cells in end-stage B16F10. H&E staining was used to determine whether BVT948 treatment was associated with increased tumor cell death. Except for B16F10, which showed a 20% reduction in viability, viability in the other tumors was not significantly changed.

### Example 6

### PTP1b Inhibition Improves Responsiveness to Chemotherapy in Multiple Preclinical Models.

The DAMP release associated with treatment naïve models may not be sufficient, with the exception of relatively smaller GEMM6 tumors, to induce macrophage inflammatory response even in the presence of BVT948. However, chemotherapy should cause a more robust release of DAMPs from dying tumor cells, thereby more substantially increasing the macrophage DAMP response. To test whether chemotherapy released DAMPs would be sufficient to activate the M1 response or whether PTP1b inhibition is necessary to rescue the response, we treated transwell co-cultured B16F10 tumor cells and macrophages with varying concentrations of Cisplatin(43, 131) in the presence or absence of PTP1b inhibitor (BVT948). Cisplatin treatment, even at 500 μM, which should induce robust tumor cell death and cause DAMP release, did not lead to substantially increased M1 associated gene expression (FIG. 6A). However, PTP1b inhibition in combination with Cisplatin treatments ranging from 50 μM to 500 μM were able to restore macrophage pro-inflammatory gene expression, in a dose dependent manner (FIG. 6A). In this model, MyD88 is described as essential for TLR signaling, which should extend to DAMP-mediated activation. To determine if this was the case, the experiment using macrophages isolated from MyD88 knockout mice was repeated. Neither increasing concentrations of Cisplatin, nor PTP1 b inhibition, were able to restore M1 gene expression in MyD88 knockout macrophages (FIG. 6B). This demonstrates that MyD88 is critical for DAMP-mediated TLR activation and the downstream pro-inflammatory response. Conversely, by the disclosed model, loss of macrophage Mer should prevent tumor-mediated suppression and allow M1 gene expression in response to tumor DAMPs. As such, Mer deficient macrophages were co-cultured with B16F10 tumor cells and found that even at 25 μM Cisplatin, lower than the 50 μM lower threshold with PTP1b inhibitor treatment, macrophages could be activated even in the presence of tumor cells (FIG. 6C).

Based on these ex vivo findings demonstrating how PTP1 b inhibition was essential to restore responsiveness to chemotherapy released DAMPs, it was next determined whether the same was also true in preclinical models. DAMPs are intrinsic to all tumors, since it was shown that tumors of diverse origins utilize MyD88 suppression to subvert the macrophage pro-inflammatory response. It was therefore assessed whether combination chemotherapy and PTP1 b inhibition could improve outcomes in preclinical models. Utilizing the B16F10, GEMM6 or LLC models, tumor bearing mice were treated with Cisplatin (B16F10, LLC) or Vemurafenib (to target the GEMM6 BrafV600E mutation) alone or in combination with PTP1b inhibition (BVT948). As a monotherapy, Cisplatin did not significantly reduce B16F10 primary tumor growth, nor did Vemurafenib for GEMM6 (FIG. 6D, 6E, respectively), indicating that both models are chemotherapy resistant. Cisplatin treatment of LLC tumors led to a modest decrease in tumor growth (30%). However, combination chemotherapy:PTP1b inhibition led to a roughly 50% decrease in tumor growth in all models, even those that were deemed chemotherapy resistant (FIGS. 6D, 6E, 6F). This demonstrates that by rescuing the macrophage DAMP response, the efficacy of chemotherapy can be surprisingly and substantially increased. As most patients will ultimately undergo chemotherapy, improving responsiveness by targeting the immune response has strong clinical implications and significant advantages over current approaches.

### Discussion of Examples 1-6

These findings reveal a conserved central mechanism, utilized by diverse tumor types (melanoma, lung, pancreatic, breast), to suppress the anti-tumor inflammatory response. Shown herein for the first time is the finding that tumor secreted Pros1 activates the macrophage Mer receptor which, in a kinase independent manner, effectively prevents TLR:MyD88-dependent M1 activation. In the tumor microenvironment, this form of immune suppression prevents a key inflammation initiating event: DAMP activation of macrophages. It is also demonstrated herein that by preventing this suppression via PTP1 b inhibition, macrophage responsiveness to tumors DAMPs is restored. In multiple preclinical models, this translates to reduced tumor growth when PTP1b inhibition is combined with chemotherapy. Excitingly, PTP1b inhibition even improves outcomes in “chemotherapy resistant” models, suggesting that tumors mask the effectiveness of chemotherapy by preventing innate immune activation.

While it cannot be excluded that PTP1b inhibition may affect other immune or stroma cells, it is demonstrated herein that in ex vivo macrophages pro-inflammatory activity is altered by this process and the overall result is a substantial reduction in tumor growth, even in chemotherapy resistant tumors.

Some of the most important implications of the present disclosure includes potential clinical impacts. Because patients with many forms of cancer do not benefit from adaptive immunotherapy, chemotherapy can be an essential treatment modality. The findings disclosed herein show that by combining PTP1b inhibition with chemotherapy can substantially reduce tumor growth leading to improved overall patient survival. These findings open the possibility of new treatments to improve one of the most frequently used therapeutics, i.e. chemotherapy, across all tumor types.

Because tumor DAMP release, rather than cancer mutational burden, drives the macrophage pro-inflammatory response, this suggests that any form of induced tumor cell death, in combination with PTP1 b inhibition, could improve the anti-tumor response. Because the disclosed combination therapy focuses on the innate immune response, it may improve the downstream adaptive anti-tumor response as well. This suggests that the disclosed combination therapy approach may also improve adaptive immune checkpoint directed therapy.

Further, even though various forms of chemotherapy have been in use for nearly 80 years, the factors dictating how well a given patient will respond are still unclear. A second implication of the present disclosure is the ability to identify susceptibility to chemotherapy using an ex vivo transwell assay. As shown in FIG. 6, chemotherapy treated tumor cells co-cultured with macrophages almost completely suppressed macrophage response to pro-inflammatory DAMPs. An assay can in some embodiments identify whether the same is true for human patient samples, which could then be correlated with patient outcomes to predict chemotherapy responsiveness.

Also from a translational perspective, the present disclosure shows that tumors can suppress most forms of TLR agonism. From an unbiased RNA-seq, it was identified that MyD88, a linchpin for TLR signaling, is suppressed by tumor-secreted Pros1. These findings highlight that this single point of regulation can have profound impacts across the entire TLR signaling family, limiting the ability to initiate pro-inflammatory signaling. As several TLR agonists are in development or clinical trials as anti-cancer therapies, it is important to know that tumors may mask their efficacy. Further, combining PTP1b inhibition with TLR agonism will further improve outcomes.

More broadly, these RNAseq findings also show that other macrophage pro-inflammatory processes (e.g., IFN signaling) are suppressed by tumor secretions. By understanding which tumor secretions and their respective downstream mechanisms causing innate suppression, new complimentary strategies to further improve cancer therapeutics can be developed.

From a basic science perspective, these results show Pros1:Mer:PTP1b:Stat1 signaling is a broadly applicable tool to effect resolution of TLR agonism and immune mitigation used in physiological wound healing or other settings. As an example, after many forms of injury, DAMPs are released. Mer has a role in preventing autoimmunity. These findings extend the role of Mer as a way to limit the immune response after injury or pathogenic infection.

Taken together, that tumors suppress the MyD88:TLR signaling axis has wide ranging implications, both translational and from a basic science perspective. The findings described herein not only optimize chemotherapy treatments and improve cancer patients' survival, but also show a new mechanism to improve adaptive immune checkpoint directed therapy responsiveness.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1. Tang J, Pearce L, O'Donnell-Tormey J, Hubbard-Lucey V M. Trends in the global immuno-oncology landscape. Nature Reviews Drug Discovery. 2018; 17(11):783-4. doi: 10.1038/nrd.2018.167.
2. Wen Z F, Liu H, Gao R, Zhou M, Ma J, Zhang Y, Zhao J, Chen Y, Zhang T, Huang F, Pan N, Zhang J, Fox B A, Hu H M, Wang L X. Tumor cell-released autophagosomes (TRAPs) promote immunosuppression through induction of M2-like macrophages with increased expression of PD-L1. J Immunother Cancer. 2018; 6(1):151. Epub 2018/12/20. doi: 10.1186/s40425-018-0452-5. PubMed PMID: 30563569; PMCID: PMC6299637.
3. Xiong H, Mittman S, Rodriguez R, Moskalenko M, Pacheco-Sanchez P, Yang Y, Nickles D, Cubas R. Anti-PD-L1 Treatment Results in Functional Remodeling of the Macrophage Compartment. Cancer Res. 2019; 79(7):1493-506. Epub 2019/01/27. doi: 10.1158/0008-5472.Can-18-3208. PubMed PMID: 30679180.
4. O'Meara T, Marczyk M, Qing T, Yaghoobi V, Blenman K, Cole K, Pelekanou V, Rimm D L, Pusztai L. Immunological Differences Between Immune-Rich Estrogen Receptor-Positive and Immune-Rich Triple-Negative Breast Cancers. JCO Precision Oncology. 2020(4):767-79. doi: 10.1200/po.19.00350.
5. Wei S C, Duffy C R, Allison J P. Fundamental Mechanisms of Immune Checkpoint Blockade Therapy. Cancer Discovery. 2018; 8(9):1069. doi: 10.1158/2159-8290.CD-18-0367.
6. Haslam A, Prasad V. Estimation of the Percentage of US Patients With Cancer Who Are Eligible for and Respond to Checkpoint Inhibitor Immunotherapy Drugs. JAMA Netw Open. 2019; 2(5):e192535. Epub 2019/05/06. doi: 10.1001/jamanetworkopen.2019.2535. PubMed PMID: 31050774; PMCID: PMC6503493.
7. Larkin J, Chiarion-Sileni V, Gonzalez R, Grob J J, Rutkowski P, Lao C D, Cowey C L, Schadendorf D, Wagstaff J, Dummer R, Ferrucci P F, Smylie M, Hogg D, Hill A, Marquez-Rodas I, Haanen J, Guidoboni M, Maio M, Schoffski P, Carlino M S, Lebbe C, McArthur G, Ascierto P A, Daniels G A, Long G V, Bastholt L, Rizzo J I, Balogh A, Moshyk A, Hodi F S, Wolchok J D. Five-Year Survival with Combined Nivolumab and Ipilimumab in Advanced Melanoma. N Engl J Med. 2019; 381(16):1535-46. Epub 2019/09/29. doi: 10.1056/NEJMoa1910836. PubMed PMID: 31562797.
8. Sharma P, Hu-Lieskovan S, Wargo J A, Ribas A. Primary, Adaptive, and Acquired Resistance to Cancer Immunotherapy. Cell. 2017; 168(4):707-23. Epub 2017/02/12. doi: 10.1016/j.cell.2017.01.017. PubMed PMID: 28187290; PMCID: PMC5391692.
9. Bell C W, Jiang W, Charles F. Reich I, Pisetsky D S. The extracellular release of HMGB1 during apoptotic cell death. American Journal of Physiology-Cell Physiology. 2006; 291(6):C1318-C25. doi: 10.1152/ajpcell.00616.2005. PubMed PMID: 16855214.
10. Dong X D, Ito N, Lotze M T, DeMarco R A, Popovic P, Shand S H, Watkins S, Winikoff S, Brown C K, Bartlett D L, Zeh H J I. High Mobility Group Box I (HMGB1) Release From Tumor Cells After Treatment: Implications for Development of Targeted Chemoimmunotherapy. Journal of Immunotherapy. 2007; 30(6):596-606. doi: 10.1097/CJI.0b013e31804efc76. PubMed PMID: 00002371-200709000-00003.
11. Tang D, Loze M T, Zeh H J, Kang R. The redox protein HMGB1 regulates cell death and survival in cancer treatment. Autophagy. 2010; 6(8):1181-3. Epub 2010/09/24. doi: 10.4161/auto.6.8.13367. PubMed PMID: 20861675.
12. Zhang X Y, Guo Z Q, Ji S Q, Zhang M, Jiang N, Li X S, Zhou L Q. Small interfering RNA targeting HMGN5 induces apoptosis via modulation of a mitochondrial pathway and Bcl-2 family proteins in prostate cancer cells. Asian J Androl. 2012; 14(3):487-92. Epub 2012/04/17. doi: 10.1038/aja.2012.18. PubMed PMID: 22504871; PMCID: PMC3720167.
13. Fauconneau B, Petegnief V, Sanfeliu C, Piriou A, Planas A M. Induction of heat shock proteins (HSPs) by sodium arsenite in cultured astrocytes and reduction of hydrogen peroxide-induced cell death. Journal of Neurochemistry. 2002; 83(6):1338-48. doi: https://doi.orq/10.1046/i.1471-4159.2002.01230.x.
14. Park Y H, Seo J H, Park J-H, Lee H S, Kim K-W. Hsp70 acetylation prevents caspase-dependent/independent apoptosis and autophagic cell death in cancer cells. Int J Oncol. 2017; 51(2):573-8. doi: 10.3892/ijo.2017.4039.
15. Son K-J, Choi K R, Ryu C-K, Lee S J, Kim H J, Lee H. Induction of immunogenic cell death of tumors by newly synthesized heterocyclic quinone derivative. PLoS One. 2017; 12(3):e0173121-e. doi: 10.1371/journal.pone.0173121. PubMed PMID: 28282460.
16. Deng L, Liang H, Fu S, Weichselbaum R R, Fu Y-X. From DNA Damage to Nucleic Acid Sensing: A Strategy to Enhance Radiation Therapy. Clinical Cancer Research. 2016; 22(1):20-5. doi: 10.1158/1078-0432.Ccr-14-3110.
17. Elkon K B. Review: Cell Death, Nucleic Acids, and Immunity: Inflammation Beyond the Grave. Arthritis Rheumatol. 2018; 70(6):805-16. Epub 2018/02/14. doi: 10.1002/art.40452. PubMed PMID: 29439290; PMCID: PMC5984680.
18. Maelfait J, Liverpool L, Rehwinkel J. Nucleic Acid Sensors and Programmed Cell Death. J Mol Biol. 2020; 432(2):552-68. Epub 2019/12/02. doi: 10.1016/j.jmb.2019.11.016. PubMed PMID: 31786265; PMCID: PMC7322524.
19. He M, Bianchi M E, Coleman T R, Tracey K J, Al-Abed Y. Exploring the biological functional mechanism of the HMGB1/TLR4/MD-2 complex by surface plasmon resonance. Mol Med. 2018; 24(1):21. Epub 2018/08/24. doi: 10.1186/s10020-018-0023-8. PubMed PMID: 30134799; PMCID: PMC6085627.
20. Urban-Wojciuk Z, Khan M M, Oyler B L, Fahraeus R, Marek-Trzonkowska N, Nita-Lazar A, Hupp T R, Goodlett D R. The Role of TLRs in Anti-cancer Immunity and Tumor Rejection. Front Immunol. 2019; 10:2388. Epub 2019/11/07. doi: 10.3389/fimmu.2019.02388. PubMed PMID: 31695691; PMCID: PMC6817561.
21. Butcher S K, O'Carroll C E, Wells C A, Carmody R J. Toll-Like Receptors Drive Specific Patterns of Tolerance and Training on Restimulation of Macrophages. Front Immunol. 2018; 9:933. Epub 2018/06/06. doi: 10.3389/fimmu.2018.00933. PubMed PMID: 29867935; PMCID: PMC5960718.
22. Gamrekelashvili J, Kapanadze T, Sablotny S, Ratiu C, Dastagir K, Lochner M, Karbach S, Wenzel P, Sitnow A, Fleig S, Sparwasser T, Kalinke U, Holzmann B, Haller H, Limbourg F P. Notch and TLR signaling coordinate monocyte cell fate and inflammation. eLife. 2020; 9:e57007. doi: 10.7554/eLife.57007.
23. Martinez A, Bono C, Megias J, Yáñez A, Gozalbo D, Gil M L. PRR signaling during in vitro macrophage differentiation from progenitors modulates their subsequent response to inflammatory stimuli. Eur Cytokine Netw. 2017; 28(3):102-10. Epub 2017/12/01. doi: 10.1684/ecn.2017.0398. PubMed PMID: 29187337.
24. Zhou L, Cao X, Fang J, Li Y, Fan M. Macrophages polarization is mediated by the combination of PRR ligands and distinct inflammatory cytokines. Int J Clin Exp Pathol. 2015; 8(9):10964-74. Epub 2015/12/01. PubMed PMID: 26617814; PMCID: PMC4637629.
25. Liu R, Liao Y Z, Zhang W, Zhou H H. Relevance of Immune Infiltration and Clinical Outcomes in Pancreatic Ductal Adenocarcinoma Subtypes. Front Oncol. 2020; 10:575264. Epub 2021/01/26. doi: 10.3389/fonc.2020.575264. PubMed PMID: 33489882; PMCID: PMC7815939.
26. Vinogradov S, Warren G, Wei X. Macrophages associated with tumors as potential targets and therapeutic intermediates. Nanomedicine (Lond). 2014; 9(5):695-707. Epub 2014/05/16. doi: 10.2217/nnm.14.13. PubMed PMID: 24827844; PMCID: PMC4149280.
27. Lin L, Chen Y-S, Yao Y-D, Chen J-Q, Chen J-N, Huang S-Y, Zeng Y-J, Yao H-R, Zeng S-H, Fu Y-S, Song E-W. CCL18 from tumor-associated macrophages promotes angiogenesis in breast cancer. Oncotarget. 2015; 6(33):34758-73. doi: 10.18632/oncotarget.5325. PubMed PMID: 26416449.
28. Petrillo M, Zannoni G F, Martinelli E, Pedone Anchora L, Ferrandina G, Tropeano G, Fagotti A, Scambia G. Polarisation of Tumor-Associated Macrophages toward M2 Phenotype Correlates with Poor Response to Chemoradiation and Reduced Survival in Patients with Locally Advanced Cervical Cancer. PLoS One. 2015; 10(9):e0136654. doi: 10.1371/journal. pone.0136654.
29. Wang J, Cao Z, Zhang X M, Nakamura M, Sun M, Hartman J, Harris R A, Sun Y, Cao Y. Novel mechanism of macrophage-mediated metastasis revealed in a zebrafish model of tumor development. Cancer Res. 2015; 75(2):306-15. Epub 2014/12/11. doi: 10.1158/0008-5472.Can-14-2819. PubMed PMID: 25492861.
30. Ricketts T D, Prieto-Dominguez N, Gowda P S, Ubil E. Mechanisms of Macrophage Plasticity in the Tumor Environment: Manipulating Activation State to Improve Outcomes. Frontiers in Immunology. 2021; 12(1565). doi: 10.3389/fimmu.2021.642285.
31. Barkal A A, Brewer R E, Markovic M, Kowarsky M, Barkal S A, Zaro B W, Krishnan V, Hatakeyama J, Dorigo O, Barkal L J, Weissman I L. CD24 signalling through macrophage Siglec-10 is a target for cancer immunotherapy. Nature. 2019; 572(7769):392-6. doi: 10.1038/s41586-019-1456-0.
32. Cheng N, Bai X, Shu Y, Ahmad 0, Shen P. Targeting tumor-associated macrophages as an antitumor strategy. Biochemical Pharmacology. 2020; 183:114354. doi: https://doi.orq/10.1016/j.bcp.2020.114354.
33. Flores-Toro J A, Luo D, Gopinath A, Sarkisian M R, Campbell J J, Charo I F, Singh R, Schall T J, Datta M, Jain R K, Mitchell D A, Harrison J K. CCR2 inhibition reduces tumor myeloid cells and unmasks a checkpoint inhibitor effect to slow progression of resistant murine gliomas. Proceedings of the National Academy of Sciences. 2020; 117(2):1129-38. doi: 10.1073/pnas.1910856117.
34. Liu X, Kwon H, Li Z, Fu Y-x. Is CD47 an innate immune checkpoint for tumor evasion?Journal of Hematology & Oncology. 2017; 10(1):12. doi: 10.1186/s13045-016-0381-z.
35. Perry C J, Munoz-Rojas A R, Meeth K M, Kellman L N, Amezquita R A, Thakral D, Du V Y, Wang J X, Damsky W, Kuhlmann A L, Sher J W, Bosenberg M, Miller-Jensen K, Kaech S M. Myeloid-targeted immunotherapies act in synergy to induce inflammation and antitumor immunity. Journal of Experimental Medicine. 2018; 215(3):877-93. doi: 10.1084/jem.20171435.
36. Ubil E, Caskey L, Holtzhausen A, Hunter D, Story C, Earp H S. Tumor-secreted Pros1 inhibits macrophage M1 polarization to reduce antitumor immune response. The Journal of Clinical Investigation. 2018; 128(6):2356-69. doi: 10.1172/JC197354.
37. Pittman K, Earp S, Ubil E. Studying the Effects of Tumor-Secreted Paracrine Ligands on Macrophage Activation using Co-Culture with Permeable Membrane Supports. JoVE. 2019(153):e60453. doi: doi:10.3791/60453.
38. Bu D, Crewe C, Kusminski C M, Gordillo R, Ghaben A L, Kim M, Park J, Deng H, Xiong W, Liu X Z, Lonning P E, Halberg N, Rios A, Chang Y, Gonzalez A, Zhang N, An Z, Scherer P E. Human endotrophin as a driver of malignant tumor growth. JCI Insight. 2019; 5. Epub 2019/03/22. doi: 10.1172/jci.insight.125094. PubMed PMID: 30896449; PMCID: PMC6538351.
39. Hussain S M, Reed L F, Krasnick B A, Miranda-Carboni G, Fields R C, Bi Y, Elahi A, Ajidahun A, Dickson P V, Deneve J L, Hawkins W G, Shibata D, Glazer E S. IL23 and TGF-ss diminish macrophage associated metastasis in pancreatic carcinoma. Sci Rep. 2018; 8(1):5808. Epub 2018/04/13. doi: 10.1038/s41598-018-24194-5. PubMed PMID: 29643359; PMCID: PMC5895618.
40. Usmani S, Sivagnanalingam U, Tkachenko O, Nunez L, Shand J C, Mullen C A. Support of acute lymphoblastic leukemia cells by nonmalignant bone marrow stromal cells. Oncol Lett. 2019; 17(6):5039-49. Epub 2019/06/13. doi: 10.3892/ol.2019.10188. PubMed PMID: 31186715; PMCID: PMC6507394.
41. Brinkley B R, Beall P T, Wible L J, Mace M L, Turner D S, Cailleau R M. Variations in cell form and cytoskeleton in human breast carcinoma cells in vitro. Cancer Res. 1980; 40(9):3118-29. Epub 1980/09/01. PubMed PMID: 7000337.
42. Sun Y, Wang X, Zhou Q, Lu Y, Zhang H, Chen Q, Zhao M, Su S. Inhibitory effect of emodin on migration, invasion and metastasis of human breast cancer MDA-MB-231 cells in vitro and in vivo. Oncol Rep. 2015; 33(1):338-46. Epub 2014/11/06. doi: 10.3892/or.2014.3585. PubMed PMID: 25370743.
43. Wang B, Zhang Y, Ye M, Wu J, Ma L, Chen H. Cisplatin-resistant MDA-MB-231 Cell-derived Exosomes Increase the Resistance of Recipient Cells in an Exosomal miR-423-5p-dependent Manner. Curr Drug Metab. 2019; 20(10):804-14. Epub 2019/08/20. doi: 10.2174/1389200220666190819151946. PubMed PMID: 31424364.
44. Hernandez C, Huebener P, Schwabe R F. Damage-associated molecular patterns in cancer: a double-edged sword. Oncogene. 2016; 35(46):5931-41. doi: 10.1038/onc.2016.104. PubMed PMID: 27086930.
45. Krysko D V, Garg A D, Kaczmarek A, Krysko O, Agostinis P, Vandenabeele P. Immunogenic cell death and DAMPs in cancer therapy. Nat Rev Cancer. 2012; 12(12):860-75. Epub 2012/11/16. doi: 10.1038/nrc3380. PubMed PMID: 23151605.
46. Krysko D V, Kaczmarek A, Krysko O, Heyndrickx L, Woznicki J, Bogaert P, Cauwels A, Takahashi N, Magez S, Bachert C, Vandenabeele P. TLR-2 and TLR-9 are sensors of apoptosis in a mouse model of doxorubicin-induced acute inflammation. Cell Death Differ. 2011; 18(8):1316-25. Epub 2011/02/12. doi: 10.1038/cdd.2011.4. PubMed PMID: 21311566; PMCID: PMC3172099.
47. Jo H, Lee J, Jeon J, Kim S Y, Chung J I, Ko H Y, Lee M, Yun M. The critical role of glucose deprivation in epithelial-mesenchymal transition in hepatocellular carcinoma under hypoxia. Sci Rep. 2020; 10(1):1538. Epub 2020/02/01. doi: 10.1038/s41598-020-58124-1. PubMed PMID: 32001727; PMCID: PMC6992695.
48. Onodera T, Momose I, Adachi H, Yamazaki Y, Sawa R, Ohba S I, Kawada M. Human pancreatic cancer cells under nutrient deprivation are vulnerable to redox system inhibition. J Biol Chem. 2020; 295(49):16678-90. Epub 2020/09/27. doi: 10.1074/jbc.RA120.013893. PubMed PMID: 32978257; PMCID: PMC7864064.
49. Osawa T, Muramatsu M, Watanabe M, Shibuya M. Hypoxia and low-nutrition double stress induces aggressiveness in a murine model of melanoma. Cancer Sci. 2009; 100(5):844-51. Epub 2009/02/18. doi: 10.1111/j.1349-7006.2009.01105.x. PubMed PMID: 19220297.
50. Demaria O, Cornen S, Daeron M, Morel Y, Medzhitov R, Vivier E. Harnessing innate immunity in cancer therapy. Nature. 2019; 574(7776):45-56. Epub 2019/10/04. doi: 10.1038/s41586-019-1593-5. PubMed PMID: 31578484.
51. Patidar A, Selvaraj S, Sarode A, Chauhan P, Chattopadhyay D, Saha B. DAMP-TLR-cytokine axis dictates the fate of tumor. Cytokine. 2018; 104:114-23. Epub 2017/10/17. doi: 10.1016/j.cyto.2017.10.004. PubMed PMID: 29032985.
52. Sims G P, Rowe D C, Rietdijk S T, Herbst R, Coyle A J. HMGB1 and RAGE in inflammation and cancer. Annu Rev Immunol. 2010; 28:367-88. Epub 2010/03/03. doi: 10.1146/annurev.immunol.021908.132603. PubMed PMID: 20192808.
53. Wang S, Zhang Y. HMGB1 in inflammation and cancer. J Hematol Oncol. 2020; 13(1):116. Epub 2020/08/25. doi: 10.1186/s13045-020-00950-x. PubMed PMID: 32831115; PMCID: PMC7443612.
54. Lord K A, Hoffman-Liebermann B, Liebermann D A. Nucleotide sequence and expression of a cDNA encoding MyD88, a novel myeloid differentiation primary response gene induced by IL6. Oncogene. 1990; 5(7):1095-7. Epub 1990/07/01. PubMed PMID: 2374694.
55. Kawai T, Adachi 0, Ogawa T, Takeda K, Akira S. Unresponsiveness of MyD88-deficient mice to endotoxin. Immunity. 1999; 11(1):115-22. Epub 1999/08/06. doi: 10.1016/s1074-7613(00)80086-2. PubMed PMID: 10435584.
56. Marr K A, Balajee S A, Hawn T R, Ozinsky A, Pham U, Akira S, Aderem A, Liles W C. Differential role of MyD88 in macrophage-mediated responses to opportunistic fungal pathogens. Infect Immun. 2003; 71(9):5280-6. Epub 2003/08/23. doi: 10.1128/IAI.71.9.5280-5286.2003. PubMed PMID: 12933875; PMCID: PMC187297.
57. Zhou S, Kurt-Jones E A, Mandell L, Cerny A, Chan M, Golenbock D T, Finberg R W. MyD88 is critical for the development of innate and adaptive immunity during acute lymphocytic choriomeningitis virus infection. Eur J Immunol. 2005; 35(3):822-30. Epub 2005/02/23. doi: 10.1002/eji.200425730. PubMed PMID: 15724245.
58. Fidler I J. Biological behavior of malignant melanoma cells correlated to their survival in vivo. Cancer Res. 1975; 35(1):218-24. Epub 1975/01/01. PubMed PMID: 1109790.
59. Fidler I J, Kripke M L. Metastasis results from preexisting variant cells within a malignant tumor. Science. 1977; 197(4306):893-5. Epub 1977/08/26. doi: 10.1126/science.887927. PubMed PMID: 887927.
60. Usary J, Darr D B, Pfefferle A D, Perou C M. Overview of Genetically Engineered Mouse Models of Distinct Breast Cancer Subtypes. Curr Protoc Pharmacol. 2016; 72:14 38 1-14 38 11. Epub 2016/03/21. doi: 10.1002/0471141755.ph1438s72. PubMed PMID: 26995547; PMCID: PMC4826719.
61. Chakraborty B, Bymerwa J, Baldi R, Liu W, Chang C-Y, McDonnell D. Abstract A44: Pharmacologic targeting of estrogen receptor in melanoma to enhance antitumor immunity. Cancer Immunol Res. 2020; 8(3 Supplement):A44-A. doi: 10.1158/2326-6074.Tumimm19-a44.
62. Bertram J S, Janik P. Establishment of a cloned line of Lewis Lung Carcinoma cells adapted to cell culture. Cancer Lett. 1980; 11(1):63-73. Epub 1980/11/01. doi: 10.1016/0304-3835(80)90130-5. PubMed PMID: 7226139.
63. Sharma S, Stolina M, Lin Y, Gardner B, Miller P W, Kronenberg M, Dubinett S M. T cell-derived IL-10 promotes lung cancer growth by suppressing both T cell and APC function. J Immunol. 1999; 163(9):5020-8. Epub 1999/10/21. PubMed PMID: 10528207.
64. Snell G D, Cloudman A M, Woodworth E. Tumor immunity in mice, induced with lyophilized tissue, as influenced by tumor strain, host strain, source of tissue, and dosage. Cancer Res. 1948; 8(9):429-37. Epub 1948/09/01. PubMed PMID: 18107384.
65. Sugiura K, Stock C C. Studies in a tumor spectrum. I. Comparison of the action of methylbis (2-chloroethyl)amine and 3-bis(2-chloroethyl)aminomethyl-4-methoxymethyl-5-hydroxy-6-methylpyridine on the growth of a variety of mouse and rat tumors. Cancer. 1952; 5(2):382-402. Epub 1952/03/01. doi: 10.1002/1097-0142(195203)5:2<382::aid-cncr2820050229>3.0.co;2-3. PubMed PMID: 14905426.
66. Guy C T, Cardiff R D, Muller W J. Induction of mammary tumors by expression of polyomavirus middle T oncogene: a transgenic mouse model for metastatic disease. Mol Cell Biol. 1992; 12(3):954-61. Epub 1992/03/01. doi: 10.1128/mcb.12.3.954-961.1992. PubMed PMID: 1312220; PMCID: PMC369527.
67. Maglione J E, Moghanaki D, Young L J, Manner C K, Ellies L G, Joseph S O, Nicholson B, Cardiff R D, MacLeod CL. Transgenic Polyoma middle-T mice model premalignant mammary disease. Cancer Res. 2001; 61(22):8298-305. Epub 2001/11/24. PubMed PMID: 11719463.
68. Clausen B E, Burkhardt C, Reith W, Renkawitz R, Forster I. Conditional gene targeting in macrophages and granulocytes using LysMcre mice. Transgenic Res. 1999; 8(4):265-77. Epub 2000/01/06. doi: 10.1023/a:1008942828960. PubMed PMID: 10621974.
69. Madisen L, Zwingman T A, Sunkin S M, Oh S W, Zariwala H A, Gu H, Ng L L, Palmiter R D, Hawrylycz M J, Jones A R, Lein E S, Zeng H. A robust and high-throughput Cre reporting and characterization system for the whole mouse brain. Nat Neurosci. 2010; 13(1):133-40. Epub 2009/12/22. doi: 10.1038/nn.2467. PubMed PMID: 20023653; PMCID: PMC2840225.
70. Bagchi A, Herrup E A, Warren H S, Trigilio J, Shin H S, Valentine C, Hellman J. MyD88-dependent and MyD88-independent pathways in synergy, priming, and tolerance between TLR agonists. J Immunol. 2007; 178(2):1164-71. Epub 2007/01/05. doi: 10.4049/jimmunol.178.2.1164. PubMed PMID: 17202381.
71. Rivas M N, Koh Y T, Chen A, Nguyen A, Lee Y H, Lawson G, Chatila T A. MyD88 is critically involved in immune tolerance breakdown at environmental interfaces of Foxp3-deficient mice. J Clin Invest. 2012; 122(5):1933-47. Epub 2012/04/03. doi: 10.1172/JC140591. PubMed PMID: 22466646; PMCID: PMC3336968.
72. Wang J Q, Jeelall Y S, Ferguson L L, Horikawa K. Toll-Like Receptors and Cancer: MYD88 Mutation and Inflammation. Frontiers in Immunology. 2014; 5(367). doi: 10.3389/fimmu.2014.00367.
73. Chung J, Uchida E, Grammer T C, Blenis J. STAT3 serine phosphorylation by ERK-dependent and -independent pathways negatively modulates its tyrosine phosphorylation. Molecular and Cellular Biology. 1997; 17(11):6508. doi: 10.1128/MCB.17.11.6508.
74. Holtzhausen A, Harris W, Ubil E, Hunter D M, Zhao J, Zhang Y, Zhang D, Liu Q, Wang X, Graham D K, Frye S V, Earp H S. TAM Family Receptor Kinase Inhibition Reverses MDSC-Mediated Suppression and Augments Anti-PD-1 Therapy in Melanoma. Cancer Immunol Res. 2019; 7(10):1672-86. Epub 2019/08/26. doi: 10.1158/2326-6066.CIR-19-0008. PubMed PMID: 31451482.
75. Lee H, Jeong A J, Ye S K. Highlighted STAT3 as a potential drug target for cancer therapy. BMB Rep. 2019; 52(7):415-23. Epub 2019/06/13. PubMed PMID: 31186087; PMCID: PMC6675244.
76. Sonnenblick A, Uziely B, Nechushtan H, Kadouri L, Galun E, Axelrod J H, Katz D, Daum H, Hamburger T, Maly B, Allweis™, Peretz T. Tumor STAT3 tyrosine phosphorylation status, as a predictor of benefit from adjuvant chemotherapy for breast cancer. Breast Cancer Res Treat. 2013; 138(2):407-13. Epub 2013/03/01. doi: 10.1007/s10549-013-2453-x. PubMed PMID: 23446809.
77. Tolomeo M, Cascio A. The Multifaced Role of STAT3 in Cancer and Its Implication for Anticancer Therapy. Int J Mol Sci. 2021; 22(2). Epub 2021/01/14. doi: 10.3390/ijms22020603. PubMed PMID: 33435349; PMCID: PMC7826746.
78. Wang Y, Shen Y, Wang S, Shen Q, Zhou X. The role of STAT3 in leading the crosstalk between human cancers and the immune system. Cancer Lett. 2018; 415:117-28. Epub 2017/12/10. doi: 10.1016/j.canlet.2017.12.003. PubMed PMID: 29222039; PMCID: PMC5748258.
79. Xiong A, Yang Z, Shen Y, Zhou J, Shen Q. Transcription Factor STAT3 as a Novel Molecular Target for Cancer Prevention. Cancers (Basel). 2014; 6(2):926-57. Epub 2014/04/20. doi: 10.3390/cancers6020926. PubMed PMID: 24743778; PMCID: PMC4074810.
80. Ait-Lounis A, Laraba-Djebari F. TNF-alpha modulates adipose macrophage polarization to M1 phenotype in response to scorpion venom. Inflamm Res. 2015; 64(11):929-36. Epub 2015/09/26. doi: 10.1007/s00011-015-0876-z. PubMed PMID: 26403661.
81. Fauskanger M, Haabeth O A W, Skjeldal F M, Bogen B, Tveita A A. Tumor Killing by CD4+ T Cells Is Mediated via Induction of Inducible Nitric Oxide Synthase-Dependent Macrophage Cytotoxicity. Frontiers in Immunology. 2018; 9(1684). doi: 10.3389/fimmu.2018.01684.
82. Mills C D, Kincaid K, Alt J M, Heilman M J, Hill A M. M-1/M-2 macrophages and the Th1/Th2 paradigm. J Immunol. 2000; 164(12):6166-73. Epub 2000/06/08. doi: 10.4049/jimmunol.164.12.6166. PubMed PMID: 10843666.
83. Niedbala W, Cai B, Liew F Y. Role of nitric oxide in the regulation of T cell functions. Ann Rheum Dis. 2006; 65 Suppl 3:iii37-40. Epub 2006/10/14. doi: 10.1136/ard.2006.058446. PubMed PMID: 17038470; PMCID: PMC1798386.
84. Vig M, Srivastava S, Kandpal U, Sade H, Lewis V, Sarin A, George A, Bal V, Durdik J M, Rath S. Inducible nitric oxide synthase in T cells regulates T cell death and immune memory. J Clin Invest. 2004; 113(12):1734-42. Epub 2004/06/17. doi: 10.1172/JC120225. PubMed PMID: 15199408; PMCID: PMC420501.
85. Wink D A, Hines H B, Cheng R Y, Switzer C H, Flores-Santana W, Vitek M P, Ridnour L A, Colton C A. Nitric oxide and redox mechanisms in the immune response. J Leukoc Biol. 2011; 89(6):873-91. Epub 2011/01/15. doi: 10.1189/jlb.1010550. PubMed PMID: 21233414; PMCID: PMC3100761.
86. Kaczanowska S, Joseph A M, Davila E. TLR agonists: our best frenemy in cancer immunotherapy. J Leukoc Biol. 2013; 93(6):847-63. Epub 2013/03/12. doi: 10.1189/jlb.1012501. PubMed PMID: 23475577; PMCID: PMC3656332.
87. Anwar M A, Shah M, Kim J, Choi S. Recent clinical trends in Toll-like receptor targeting therapeutics. Med Res Rev. 2019; 39(3):1053-90. Epub 2018/11/20. doi: 10.1002/med.21553. PubMed PMID: 30450666; PMCID: PMC6587958.
88. Durbin J E, Hackenmiller R, Simon M C, Levy D E. Targeted disruption of the mouse Stat1 gene results in compromised innate immunity to viral disease. Cell. 1996; 84(3):443-50. Epub 1996/02/09. doi: 10.1016/s0092-8674(00)81289-1. PubMed PMID: 8608598.
89. Khodarev N N, Roizman B, Weichselbaum R R. Molecular Pathways: Interferon/Stat1 Pathway: Role in the Tumor Resistance to Genotoxic Stress and Aggressive Growth. Clinical Cancer Research. 2012; 18(11):3015-21. doi: 10.1158/1078-0432.Ccr-11-3225.
90. Murphy K M, Ouyang W, Szabo S J, Jacobson N G, Guler M L, Gorham J D, Gubler U, Murphy T L. T helper differentiation proceeds through Stat1-dependent, Stat4-dependent and Stat4-independent phases. Curr Top Microbiol Immunol. 1999; 238:13-26. Epub 1999/03/24. doi: 10.1007/978-3-662-09709-O_2. PubMed PMID: 10087648.
91. Ohmori Y, Hamilton T A. Requirement for STAT1 in LPS-induced gene expression in macrophages. J Leukoc Biol. 2001; 69(4):598-604. Epub 2001/04/20. PubMed PMID: 11310846.
92. Srivastava R M, Trivedi S, Concha-Benavente F, Hyun-Bae J, Wang L, Seethala R R, Branstetter BFt, Ferrone S, Ferris R L. STAT1-Induced HLA Class I Upregulation Enhances Immunogenicity and Clinical Response to Anti-EGFR mAb Cetuximab Therapy in HNC Patients. Cancer Immunol Res. 2015; 3(8):936-45. Epub 2015/05/15. doi: 10.1158/2326-6066.CIR-15-0053. PubMed PMID: 25972070; PMCID: PMC4526378.
93. Yamaoka K, Otsuka T, Niiro H, Nakashima H, Tanaka Y, Nagano S, Ogami E, Niho Y, Hamasaki N, Izuhara K. Selective DNA-binding activity of interleukin-10-stimulated STAT molecules in human monocytes. J Interferon Cytokine Res. 1999; 19(6):679-85. Epub 1999/08/05. doi: 10.1089/107999099313839. PubMed PMID: 10433370.
94. Serezani C H, Lewis C, Jancar S, Peters-Golden M. Leukotriene B4 amplifies NF-kappaB activation in mouse macrophages by reducing SOCS1 inhibition of MyD88 expression. J Clin Invest. 2011; 121(2):671-82. Epub 2011/01/06. doi: 10.1172/JC143302. PubMed PMID: 21206089; PMCID: PMC3026722.
95. Feng Z, Zheng W, Tang Q, Cheng L, Li H, Ni W, Pan X. Fludarabine inhibits STAT1-mediated up-regulation of caspase-3 expression in dexamethasone-induced osteoblasts apoptosis and slows the progression of steroid-induced avascular necrosis of the femoral head in rats. Apoptosis. 2017; 22(8):1001-12. Epub 2017/06/12. doi: 10.1007/s10495-017-1383-1. PubMed PMID: 28601953.
96. Frank D A, Mahajan S, Ritz J. Fludarabine-induced immunosuppression is associated with inhibition of STAT1 signaling. Nat Med. 1999; 5(4):444-7. Epub 1999/04/15. doi: 10.1038/7445. PubMed PMID: 10202937.
97. Bohmer F D, Friedrich K. Protein tyrosine phosphatases as wardens of STAT signaling. JAKSTAT. 2014; 3(1):e28087. Epub 2014/04/30. doi: 10.4161/jkst.28087. PubMed PMID: 24778927; PMCID: PMC3995736.
98. Pike K A, Hutchins A P, Vinette V, Theberge J F, Sabbagh L, Tremblay M L, Miranda-Saavedra D. Protein tyrosine phosphatase 1B is a regulator of the interleukin-10-induced transcriptional program in macrophages. Sci Signal. 2014; 7(324):ra43. Epub 2014/05/08. doi: 10.1126/scisignal.2005020. PubMed PMID: 24803538.
99. Yue L, Yan M, Chen S, Cao H, Li H, Xie Z. PTP1B negatively regulates STAT1-independent Pseudomonas aeruginosa killing by macrophages. Biochem Biophys Res Commun. 2020; 533(3):296-303. Epub 2020/09/23. doi: 10.1016/j.bbrc.2020.09.032. PubMed PMID: 32958258.
100. Al Kafri N, Hafizi S. Galectin-3 Stimulates Tyro3 Receptor Tyrosine Kinase and Erk Signalling, Cell Survival and Migration in Human Cancer Cells. Biomolecules. 2020; 10(7):1035. doi: 10.3390/biom10071035. PubMed PMID: 32664510.
101. Al Kafri N, Hafizi S. Tumour-Secreted Protein S (ProS1) Activates a Tyro3-Erk Signalling Axis and Protects Cancer Cells from Apoptosis. Cancers (Basel). 2019; 11(12). Epub 2019/11/27. doi: 10.3390/cancers11121843. PubMed PMID: 31766614; PMCID: PMC6966665.
102. Graham D K, DeRyckere D, Davies K D, Earp H S. The TAM family: phosphatidylserine sensing receptor tyrosine kinases gone awry in cancer. Nat Rev Cancer. 2014; 14(12):769-85. Epub 2015/01/09. doi: 10.1038/nrc3847. PubMed PMID: 25568918.
103. Kasikara C, Davra V, Calianese D, Geng K, Spires T E, Quigley M, Wichroski M, Sriram G, Suarez-Lopez L, Yaffe M B, Kotenko S V, De Lorenzo M S, Birge R B. Pan-TAM Tyrosine Kinase Inhibitor BMS-777607 Enhances Anti-PD-1 mAb Efficacy in a Murine Model of Triple-Negative Breast Cancer. Cancer Research. 2019; 79(10):2669. doi: 10.1158/0008-5472.CAN-18-2614.
104. Lemke G. Biology of the TAM receptors. Cold Spring Harb Perspect Biol. 2013; 5(11):a009076-a. doi: 10.1101/cshperspect.a009076. PubMed PMID: 24186067.
105. Caberoy N B, Zhou Y, Li W. Tubby and tubby-like protein 1 are new MerTK ligands for phagocytosis. EMBO J. 2010; 29(23):3898-910. Epub 2010/10/26. doi: 10.1038/emboj.2010.265. PubMed PMID: 20978472.
106. Cook R S, Jacobsen K M, Wofford A M, DeRyckere D, Stanford J, Prieto A L, Redente E, Sandahl M, Hunter D M, Strunk K E, Graham D K, Earp H S, 3rd. MerTK inhibition in tumor leukocytes decreases tumor growth and metastasis. J Clin Invest. 2013; 123(8):3231-42. Epub 2013/07/23. doi: 10.1172/jci67655. PubMed PMID: 23867499; PMCID: PMC3726162.
107. Guttridge K L, Luft J C, Dawson T L, Kozlowska E, Mahajan N P, Varnum B, Earp H S. Mer receptor tyrosine kinase signaling: prevention of apoptosis and alteration of cytoskeletal architecture without stimulation or proliferation. J Biol Chem. 2002; 277(27):24057-66. Epub 2002/04/04. doi: 10.1074/jbc.M112086200. PubMed PMID: 11929866.
108. Nishi C, Yanagihashi Y, Segawa K, Nagata S. MERTK tyrosine kinase receptor together with TIM4 phosphatidylserine receptor mediates distinct signal transduction pathways for efferocytosis and cell proliferation. Journal of Biological Chemistry. 2019; 294(18):7221-30. doi: https://doi.orq/10.1074/jbc.RA118.006628.
109. Scott R S, McMahon E J, Pop S M, Reap E A, Caricchio R, Cohen P L, Earp H S, Matsushima G K. Phagocytosis and clearance of apoptotic cells is mediated by MER. Nature. 2001; 411(6834):207-11. Epub 2001/05/11. doi: 10.1038/35075603. PubMed PMID: 11346799.
110. Yanagihashi Y, Segawa K, Maeda R, Nabeshima Y I, Nagata S. Mouse macrophages show different requirements for phosphatidylserine receptor Tim4 in efferocytosis. Proc Natl Acad Sci USA. 2017; 114(33):8800-5. Epub 2017/08/05. doi: 10.1073/pnas.1705365114. PubMed PMID: 28768810; PMCID: PMC5565444.
111. Felip E, Brunsvig P, Vinolas N, Aix S P, Costa E C, Gomez M D, Perez J M T, Arriola E, Campelo R G, Spicer J F, Thompson J R, Granados A L O, Holt R J, Lorens K, Lorens J B, Shoaib M, Siddiqui A, Schmidt E V, Chisamore M J, Krebs M. A phase II study of bemcentinib (BGB324), a first-in-class highly selective AXL inhibitor, with pembrolizumab in pts with advanced NSCLC: OS for stage I and preliminary stage II efficacy. Journal of Clinical Oncology. 2019; 37(15_suppl):9098-. doi: 10.1200/JCO.2019.37.15_suppl.9098.
112. Holland S J, Pan A, Franci C, Hu Y, Chang B, Li W, Duan M, Torneros A, Yu J, Heckrodt T J, Zhang J, Ding P, Apatira A, Chua J, Brandt R, Pine P, Goff D, Singh R, Payan D G, Hitoshi Y. R428, a selective small molecule inhibitor of Axl kinase, blocks tumor spread and prolongs survival in models of metastatic breast cancer. Cancer Res. 2010; 70(4):1544-54. Epub 2010/02/11. doi: 10.1158/0008-5472.Can-09-2997. PubMed PMID: 20145120.
113. Lorens J, Arce-Lara C E, Arriola E, Brunsvig P, Costa E C, Domine M, Dragnev K H, Felip E, Campelo R G, Krebs M, Aix S P, Spicer J F, Perez J M T, Vinolas N, Holt R J, Brown A, Chisamore M J. Phase II open-label, multi-centre study of bemcentinib (BGB324), a first-in-class selective AXL inhibitor, in combination with pembrolizumab in patients with advanced NSCLC. Journal of Clinical Oncology. 2018; 36(15_suppl):3078-. doi: 10.1200/JCO.2018.36.15_suppl.3078.
114. Myers K V, Amend S R, Pienta K J. Targeting Tyro3, Axl and MerTK (TAM receptors): implications for macrophages in the tumor microenvironment. Mol Cancer. 2019; 18(1):94. Epub 2019/05/16. doi: 10.1186/s12943-019-1022-2. PubMed PMID: 31088471; PMCID: PMC6515593.
115. Paolino M, Choidas A, Wallner S, Pranjic B, Uribesalgo I, Loeser S, Jamieson A M, Langdon W Y, Ikeda F, Fededa J P, Cronin S J, Nitsch R, Schultz-Fademrecht C, Eickhoff J, Menninger S, Unger A, Torka R, Gruber T, Hinterleitner R, Baier G, Wolf D, Ullrich A, Klebl B M, Penninger J M. The E3 ligase Cbl-b and TAM receptors regulate cancer metastasis via natural killer cells. Nature. 2014; 507(7493):508-12. Epub 2014/02/21. doi: 10.1038/nature12998. PubMed PMID: 24553136; PMCID: PMC6258903.
116. Pontara A, Paolino G, Gregorc V, Mercuri S R, Bulotta A, Bearzi P, Doglioni C, Rizzo N. Immunohistochemical Evaluation and Clinicopathological Correlation of Mer and Axl Tyrosine Kinase TAM Receptors in Cutaneous Melanoma. Dermatol Pract Concept. 2020; 10(2):e2020029. Epub 2020/05/05. doi: 10.5826/dpc.1002a29. PubMed PMID: 32363092; PMCID: PMC7190557.
117. Maree A O, Jneid H, Palacios I F, Rosenfield K, MacRae CA, Fitzgerald D J. Growth arrest specific gene (GAS) 6 modulates platelet thrombus formation and vascular wall homeostasis and represents an attractive drug target. Curr Pharm Des. 2007; 13(26):2656-61. Epub 2007/09/28. doi: 10.2174/138161207781662948. PubMed PMID: 17897008.
118. Prieto A L, Weber J L, Tracy S, Heeb M J, Lai C. Gas6, a ligand for the receptor protein-tyrosine kinase Tyro-3, is widely expressed in the central nervous system. Brain Res. 1999; 816(2):646-61. Epub 1999/01/08. doi: 10.1016/s0006-8993(98)01159-7. PubMed PMID: 9878891.
119. Wu G, Ma Z, Hu W, Wang D, Gong B, Fan C, Jiang S, Li T, Gao J, Yang Y. Molecular insights of Gas6/TAM in cancer development and therapy. Cell Death Dis. 2017; 8(3):e2700. Epub 2017/03/24. doi: 10.1038/cddis.2017.113. PubMed PMID: 28333143; PMCID: PMC5386520.
120. Lew E D, Oh J, Burrola P G, Lax I, Zagórska A, Través PG, Schlessinger J, Lemke G. Differential TAM receptor-ligand-phospholipid interactions delimit differential TAM bioactivities. eLife. 2014; 3:e03385. doi: 10.7554/eLife.03385.
121. Caberoy N B, Maiguel D, Kim Y, Li W. Identification of tubby and tubby-like protein 1 as eat-me signals by phage display. Exp Cell Res. 2010; 316(2):245-57. Epub 2009/10/20. doi: 10.1016/j.yexcr.2009.10.008. PubMed PMID: 19837063; PMCID: PMC2789476.
122. Mukhopadhyay S, Jackson P K. The tubby family proteins. Genome Biol. 2011; 12(6):225. Epub 2011/07/05. doi: 10.1186/gb-2011-12-6-225. PubMed PMID: 21722349; PMCID: PMC3218838.
123. Li L C, Li J, Gao J. Functions of galectin-3 and its role in fibrotic diseases. J Pharmacol Exp Ther. 2014; 351(2):336-43. Epub 2014/09/07. doi: 10.1124/jpet.114.218370. PubMed PMID: 25194021.
124. MacKinnon AC, Farnworth S L, Hodkinson P S, Henderson N C, Atkinson K M, Leffler H, Nilsson U J, Haslett C, Forbes S J, Sethi T. Regulation of alternative macrophage activation by galectin-3. Journal of immunology (Baltimore, Md 1950). 2008; 180(4):2650-8. doi: 10.4049/jimmunol.180.4.2650. PubMed PMID: 18250477.
125. Amarante-Mendes G P, Adjemian S, Branco L M, Zanetti L C, Weinlich R, Bortoluci K R. Pattern Recognition Receptors and the Host Cell Death Molecular Machinery. Frontiers in Immunology. 2018; 9(2379). doi: 10.3389/fimmu.2018.02379.
126. Aubrey B J, Kelly G L, Janic A, Herold M J, Strasser A. How does p53 induce apoptosis and how does this relate to p53-mediated tumour suppression?Cell Death Differ. 2018; 25(1):104-13. Epub 2017/11/18. doi: 10.1038/cdd.2017.169. PubMed PMID: 29149101; PMCID: PMC5729529.
127. Rock K L, Kono H. The inflammatory response to cell death. Annu Rev Pathol. 2008; 3:99-126. Epub 2007/11/28. doi: 10.1146/annurev.pathmechdis.3.121806.151456. PubMed PMID: 18039143; PMCID: PMC3094097.
128. Roos W P, Thomas A D, Kaina B. DNA damage and the balance between survival and death in cancer biology. Nature reviews Cancer. 2016; 16(1):20-33. doi: 10.1038/nrc.2015.2. PubMed PMID: 26678314.
129. Solari J I G, Filippi-Chiela E, Pilar E S, Nunes V, Gonzalez E A, Figueiró F, Andrade C F, Klamt F. Damage-associated molecular patterns (DAMPs) related to immunogenic cell death are differentially triggered by clinically relevant chemotherapeutics in lung adenocarcinoma cells. BMC Cancer. 2020; 20(1):474. Epub 2020/05/28. doi: 10.1186/s12885-020-06964-5. PubMed PMID: 32456685; PMCID: PMC7251700.
130. Perez-Guijarro E, Day C P, Merlino G, Zaidi M R. Genetically engineered mouse models of melanoma. Cancer. 2017; 123(S11):2089-103. Epub 2017/05/26. doi: 10.1002/cncr.30684. PubMed PMID: 28543694; PMCID: PMC5445933.
131. Sommariva M, de Cesare M, Meini A, Cataldo A, Zaffaroni N, Tagliabue E, Balsari A. High efficacy of CpG-ODN, Cetuximab and Cisplatin combination for very advanced ovarian xenograft tumors. J Transl Med. 2013; 11(1):25. doi: 10.1186/1479-5876-11-25.
132. Xu X, Wang X, Guo Y, Bai Y, He S, Wang N, Lin Y, Fisher M, Liu Q, Yao Y. Inhibition of PTP1B Promotes M2 Polarization via MicroRNA-26a/MKP1 Signaling Pathway in Murine Macrophages. Front Immunol. 2019; 10:1930. Epub 2019/09/03. doi: 10.3389/fimmu.2019.01930. PubMed PMID: 31474996; PMCID: PMC6702542.
133. Carmona S, Brunel J M, Bonier R, Sbarra V, Robert S, Borentain P, Lombardo D, Mas E, Gerolami R. A squalamine derivative, NV669, as a novel PTP1B inhibitor: in vitro and in vivo effects on pancreatic and hepatic tumor growth. Oncotarget. 2019; 10(62):6651-67. Epub 2019/12/06. doi: 10.18632/oncotarget.27286. PubMed PMID: 31803360; PMCID: PMC6877102.
134. Kumar G S, Page R, Peti W. The mode of action of the Protein tyrosine phosphatase 1B inhibitor Ertiprotafib. PLoS One. 2020; 15(10):e0240044. Epub 2020/10/03. doi: 10.1371/journal.pone.0240044. PubMed PMID: 33007022; PMCID: PMC7531832.
135. Ray A, Dittel B N. Isolation of mouse peritoneal cavity cells. J Vis Exp. 2010(35). Epub 2010/01/30. doi: 10.3791/1488. PubMed PMID: 20110936; PMCID: PMC3152216.
136. Livak K J, Schmittgen T D. Analysis of relative gene expression data using real-time quantitative PCR and the 2(−Delta Delta C(T)) Method. Methods. 2001; 25(4):402-8. Epub 2002/02/16. doi: 10.1006/meth.2001.1262. PubMed PMID: 11846609.
137. Cai L, Rothbart S B, Lu R, Xu B, Chen W Y, Tripathy A, Rockowitz S, Zheng D, Patel D J, Allis C D, Strahl B D, Song J, Wang G G. An H3K36 methylation-engaging Tudor motif of polycomb-like proteins mediates PRC2 complex targeting. Mol Cell. 2013; 49(3):571-82. Epub 2013/01/01. doi: 10.1016/j.molcel.2012.11.026. PubMed PMID: 23273982; PMCID: PMC3570589.
138. Lu R, Wang P, Parton T, Zhou Y, Chrysovergis K, Rockowitz S, Chen W Y, Abdel-Wahab O, Wade P A, Zheng D, Wang G G. Epigenetic Perturbations by Arg882-Mutated DNMT3A Potentiate Aberrant Stem Cell Gene-Expression Program and Acute Leukemia Development. Cancer Cell. 2016; 30(1):92-107. Epub 2016/06/28. doi: 10.1016/j.ccell.2016.05.008.
PubMed PMID: 27344947; PMCID: PMC4945461. 139. The Merck Manual of Diagnosis and Therapy, 15th 35 Ed., Berkow et al., eds., 1987, Rahway, N.J.,

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A pharmaceutical composition, the pharmaceutical composition comprising:
one or more compounds inhibiting PTP1 b activity; and
one or more chemotherapeutic agents.

2. The pharmaceutical composition of claim 1, wherein the PTP1b inhibitor comprises a small molecule, optionally wherein the PTP1b inhibitor comprises an anti-sense inhibitor, an allosteric/reversible inhibitor, and/or a permanent/chemical modification inhibitor.

3. The pharmaceutical composition of any of claims 1 to 2, wherein the PTP1b inhibitor is selected from the group consisting of BVT948, PTP1b inhibitor III, NSC87877, PTP1b inhibitor, PTP Inhibitor IV, MSI-1436 and ISIS-PTP1B_{RX}.

4. The pharmaceutical composition of any of claims 1 to 3, wherein the PTP1b inhibitor is BVT948.

5. The pharmaceutical composition of any of claims 1 to 4, wherein PTP1b inhibition by one or more compounds inhibiting PTP1b activity substantially prevents Pros1:Mer:Stat1-mediated suppression of MyD88 expression, optionally wherein the Pros1:Mer:Stat1-mediated suppression of MyD88 expression is prevented at least about 40% to at least about 99%.

6. The pharmaceutical composition of any of claims 1 to 5, wherein the composition rescues the macrophage M1 response to tumor DAMPs due to PTP1b inhibition.

7. The pharmaceutical composition of any of claims 1 to 6, wherein the one or more chemotherapeutic agents comprise an alkylating agent, a nitrosourea, an anti-metabolite, a plant alkaloid/natural product, an anti-tumor antibiotic and/or combination thereof, optionally wherein the chemotherapeutic agent is selected from the group consisting of daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxu-ridine (5-FUdR), methotrexate (MTX), colchicine, Vincristine, vinblastine, etoposide, teniposide, cisplatin and diethylstilbestrol (DES).

8. The pharmaceutical composition of any of claims 1 to 7, wherein the composition improves a patient's responsiveness to chemotherapy upon administration to a patient.

9. The pharmaceutical composition of any of claims 1 to 8, wherein the composition is configured to provide a co-treatment of tumors and cancers with chemotherapy and PTP1 b inhibition to restore the macrophage DAMP pro-inflammatory response.

10. The pharmaceutical composition of any of claims 1 to 9, further comprising a pharmaceutically acceptable carrier, diluent, enhancer, excipient, and/or combinations thereof.

11. The pharmaceutical composition of any of claims 1 to 10, further comprising a checkpoint blockade component, optionally wherein the checkpoint blockade component comprises an anti-PD-1, anti-PD-L1, or anti-CTLA4 component, or combination thereof.

12. A method of treating a subject, the method comprising administering to a subject in need of treatment a pharmaceutical composition comprising:
one or more compounds inhibiting PTP1 b activity; and
one or more chemotherapeutic agents.

13. The method of claim 12, wherein the subject is a subject suffering from a cancer or tumor, optionally where the subject is suffering from a cancer or tumor type selected from the group consisting of melanoma, lung cancer, pancreatic cancer and/or breast cancer.

14. The method of any of claims 12-13, wherein the subject is a human subject, optionally wherein the subject is a human subject suffering from a chemotherapy resistant tumor or cancer.

15. The method of any of claims 12-14, wherein the PTP1b inhibitor comprises a small molecule, optionally wherein the PTP1b inhibitor comprises an anti-sense inhibitor, an allosteric/reversible inhibitor, and/or a permanent/chemical modification inhibitor.

16. The method of any of claims 12-15, wherein the PTP1b inhibitor is selected from the group consisting of BVT948, PTP1b inhibitor III, NSC87877, PTP1b inhibitor, PTP Inhibitor IV, MSI-1436 and ISIS-PTP1B_{RX}.

17. The method of claim 16, wherein the PTP1b inhibitor is BVT948.

18. The method of any of claims 12-17, wherein PTP1b inhibition by one or more compounds inhibiting PTP1b activity substantially prevents Pros1:Mer:Stat1-mediated suppression of MyD88 expression, optionally wherein the Pros1:Mer:Stat1-mediated suppression of MyD88 expression is prevented at least about 40% to at least about 99%.

19. The method of any of claims 12-18, wherein the composition rescues the macrophage M1 response to tumor DAMPs due to PTP1 b inhibition.

20. The method of any of claims 12-19, wherein the one or more chemotherapeutic agents comprise an alkylating agent, a nitrosourea, an anti-metabolite, a plant alkaloid/natural product, an anti-tumor antibiotic and/or combination thereof, optionally wherein the chemotherapeutic agent is selected from the group consisting of daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxu-ridine (5-FUdR), methotrexate (MTX), colchicine, Vincristine, vinblastine, etoposide, teniposide, cisplatin and diethylstilbestrol (DES).

21. The method of any of claims 12-20, wherein the method substantially prevents in the subject a suppression of an immune response by PTP1 b inhibition, and wherein macrophage responsiveness to tumors DAMPs is restored, optionally wherein the prevention in the suppression of an immune response by PTP1 b is about 40% to about 100%.

22. The method of any of claims 12-21, wherein the PTP1b inhibitor is co-administered with the chemotherapeutic agent.

23. The method of any of claims 12-22, wherein the co-administration of the PTP1 b inhibitor with the chemotherapeutic agent has a synergistic effect causing an improved outcome as compared to the administration of the PTP1 b inhibitor or the chemotherapeutic agent alone, optionally wherein the improved outcome comprises an about 25% to about 100% reduction in tumor growth relative to the administration of the PTP1b inhibitor or the chemotherapeutic agent alone.

24. The method of any of claims 12-23, wherein administration of the PTP1 b inhibitor improves the patient's responsiveness to chemotherapy, optionally an improvement of at least 50% as compared to chemotherapy without PTP1b inhibition.

25. The method of any of claims 12-24, wherein administration of the PTP1 b inhibitor improves the patient's responsiveness to chemotherapy, wherein the patient is suffering from a chemotherapy resistant tumor or cancer.

26. A method of restoring an anti-tumor immune response in a subject receiving chemotherapy, the method comprising administering to the subject a PTP1 b inhibitor prior to, during and/or after receiving a chemotherapeutic agent, wherein an anti-tumor immune response in the subject is restored and/or enhanced.

27. The method of claim 26, wherein the subject is a subject suffering from a cancer or tumor, optionally a subject suffering from a tumor or cancer type selected from the group consisting of melanoma, lung cancer, pancreatic cancer and/or breast cancer.

28. The method of any of claims 26-27, wherein the chemotherapeutic agent comprises an alkylating agent, a nitrosourea, an anti-metabolite, a plant alkaloid/natural product, an anti-tumor antibiotic and/or combination thereof, optionally wherein the chemotherapeutic agent is selected from the group consisting of daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxu-ridine (5-FUdR), methotrexate (MTX), colchicine, Vincristine, vinblastine, etoposide, teniposide, cisplatin and diethylstilbestrol (DES).

29. The method of any of claims 26-28, wherein administration of the PTP1 b inhibitor improves the subject's responsiveness to the chemotherapy, optionally an improvement of at least about 50% as compared to chemotherapy without PTP1 b inhibition.

30. The method of claim 26, further comprising administering to the subject a checkpoint blockade component, optionally wherein the checkpoint blockade component comprises an anti-PD-1, anti-PD-L1, or anti-CTLA4 component, or combination thereof.
